(19) United States
(12) Patent Application Publication
(10) Pub. No.: US 2025/0312339 A1
(43) Pub. Date: Oct. 9, 2025

(54) METHODS AND COMPOSITIONS COMPRISING A SHP2 INHIBITOR AND A PD-LI BINDING ANTAGONIST

(71) Applicant: Genentech, Inc., South San Francisco, CA (US)

(72) Inventors: Jennifer Eng-Wong, San Carlos, CA (US); Pablo Saenz-Lopez Larrocha, San Francisco, CA (US); Jeffrey Lau, South San Francisco, CA (US); Nicole Sodir, San Bruno, CA (US); Joanne Irene Adamkewicz, Belmont, CA (US); Danilo Maddalo, Kensington, CA (US); Mark Andrew Merchant, Redwood City, CA (US); Gaurav Pathria, Burlingame, CA (US)

(21) Appl. No.: 18/864,984

(22) PCT Filed: May 12, 2023
(86) PCT No.: PCT/US2023/066920

§ 371 Date: Nov. 12, 2024

Related U.S. Application Data
(60) Provisional application No. 63/341,361, filed on May 12, 2022.

(51) Int. Cl. A61K 31/4985; A61K 39/00; A61K 39/395; A61P 35/00

(52) U.S. Cl. CPC A61K 31/4985; A61K 39/3955; A61P 35/00; A61K 2039/505; A61K 2039/545

(57) ABSTRACT

Provided herein are combination therapies comprising a SHP2 inhibitor (e.g., Compound 1) and a PD-L1 binding antagonist (e.g., atezolizumab) and methods of using such combination therapies.

### TECHNICAL FIELD

Provided herein are combination therapies comprising a SHP2 inhibitor (e.g., Compound 1) and a PD-L1 binding antagonist (e.g., atezolizumab) and methods of using such combination therapies.

### SEQUENCE LISTING

This application incorporates by reference the Computer Readable Form (CRF) of a Sequence Listing in ASCII text format submitted via EFS-Web. The Sequence Listing text file submitted via EFS-Web, entitled P37266_SEQ_LISTING_ST26.txt, with a file size of 18 kilobytes, created on Apr. 25, 2023.

### BACKGROUND

Protein tyrosine phosphorylation plays a central role in the pathogenesis of cancer. SSHP2 (Src homology-2 domain-containing protein tyrosine phosphatase-2) is a protein tyrosine phosphatase encoded by PTPN11 gene; it serves as a central node for several intracellular oncogenic signaling pathways such as RAS/Raf/MAPK, PI3K/AKT, Jak/STAT and PD-1/PDL-1 pathways (J Med Chem. 2020; 63: 11368-11396). Gain of function mutations and/or overexpression of SHP2 has been associated with genetic developmental diseases (such as Noonan Syndrome) and various types cancers (such as breast cancer, melanoma, non-small-cell lung adenocarcinoma (NSCL), and others (Eur J Med Genet. 2015; 58(10): 509-25; Pharmacol Res. 2020; 152: 104595-104605). Under basal conditions, SHP2 exists in an auto-inhibited conformation in which the catalytic activity of SHP2 is suppressed. Somatic missense mutations in SHP2, which have been detected in leukemias and more rarely in solid tumors, destabilize the auto-inhibited conformation of SHP2, thereby resulting in aberrant hyperactivation.

Thus, there is a need for effective therapies and combination therapies for treating cancers such as lung cancer, head and neck cancer, and other solid tumors.

### SUMMARY OF THE DISCLOSURE

Provided herein are solutions to these and other problems in the art.

In one aspect provided herein is a combination therapy comprising (a) Compound 1, or a pharmaceutically acceptable salt thereof, as described herein, and (b) a PD-L1 binding antagonist as described herein.

In another aspect provided herein is a method of treating lung cancer, head and neck cancer, or melanoma in a patient in need thereof, the method comprising administering during a treatment period an effective amount of a combination therapy comprising (a) Compound 1, or a pharmaceutically acceptable salt thereof, and (b) a PD-L1 binding antagonist, as described herein. In some embodiments, the lung cancer is non-small cell lung cancer (NSCLC). In some embodiments, the head and neck cancer is head and neck squamous cell carcinoma (HNSCC). In some embodiments, the melanoma is BRAF Wild Type (WT) melanoma.

In another aspect provided herein is a method of treating lung cancer, head and neck cancer, or melanoma in a patient in need thereof, the method comprising administering to the patient a treatment regimen comprising an effective amount of Compound 1, or a pharmaceutically acceptable salt thereof, and a PD-L1 binding antagonist, as described herein. In some embodiments, the lung cancer is non-small cell lung cancer (NSCLC). In some embodiments, the head and neck cancer is head and neck squamous cell carcinoma (HNSCC). In some embodiments, the melanoma is BRAF Wild Type (WT) melanoma.

In still another aspect provided herein is use (U1) of a combination therapy comprising Compound 1, or a pharmaceutically acceptable salt thereof, and atezolizumab, as described herein, for the treatment of lung cancer, head and neck cancer, or melanoma as described herein. In some embodiments, the lung cancer is non-small cell lung cancer (NSCLC). In some embodiments, the head and neck cancer is head and neck squamous cell carcinoma (HNSCC). In some embodiments, the melanoma is BRAF Wild Type (WT) melanoma.

In still another aspect provided herein is use (U5) of a combination therapy comprising Compound 1, or a pharmaceutically acceptable salt thereof, and atezolizumab, as described herein, for the manufacture of a medicament for the treatment of lung cancer, head and neck cancer, or melanoma. In some embodiments, the lung cancer is non-small cell lung cancer (NSCLC). In some embodiments, the head and neck cancer is head and neck squamous cell carcinoma (HNSCC). In some embodiments, the melanoma is BRAF Wild Type (WT) melanoma.

In still another aspect provided herein is a combination therapy comprising Compound 1, or a pharmaceutically acceptable salt thereof, and atezolizumab, as described herein, for use in the treatment of lung cancer, head and neck cancer, or melanoma. In some embodiments, the lung cancer is non-small cell lung cancer (NSCLC). In some embodiments, the head and neck cancer is head and neck squamous cell carcinoma (HNSCC). In some embodiments, the melanoma is BRAF Wild Type (WT) melanoma.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates the effect on individual tumor volumes of Compound 1 and anti-PD-L1 Combination Therapy on the EMT6 Mammary Carcinoma in BALB/c Mice.

FIG. 2 illustrates the growth contrast analysis for the Compound 1 and anti-PD-L1 combination Therapy on the EMT6 Mammary Carcinoma in BALB/c Mice.

FIG. 3 illustrates individual tumor AUC-based growth for the Compound 1 and Anti-PD-L1 Combination Therapy on the EMT6 Mammary Carcinoma in BALB/c Mice.

FIG. 4A illustrates the EMT6 individual tumor volume and MHC-I Expression after seven days of treatment.

FIG. 4B illustrates Compound 1+/− anti-PD-L1 group treatments increase the MFI of MHC-I and PD-L1 in tumors versus the vehicle group after seven days of treatment (MHC-I (p=0.0354, p=0.0044) PD-L1 (p=0.0127, p=0.0018)).

FIG. 5A illustrates the TAM population in the tumor microenvironment was reduced in Compound 1+/− anti-PD-L1 group treatments in comparison to the vehicle group (P=0.0072, P=0.0098). MDSC were reduced in the tumor microenvironment of mice treated with Compound 1+/− anti-PD-L1 group treatments versus vehicle group (p=0.0058, p=0.0083).

FIG. 5B illustrates the frequency of CD8 T cell population was increased in the tumor microenvironment of mice treated with Compound 1+/− anti-PD-L1 group treatments in comparison to the vehicle group (P=0.0058, p=0.008). The frequency of CD4 T cell population and NK cells were increased in the tumor microenvironment of mice that were treated with Compound 1+/− anti-PD-L1 in comparison to the vehicle group (CD4 (p=0.0067, p=0.002) and NK (p=0.013, p=0.024).

FIG. 6A illustrates there was an increase in Frequency of CD8 population expressing IFNg or TNFa in the Compound 1+/− anti-PD-L1 group treatments in comparison to vehicle group (IFNg (p=0.0002, p=0.0001), TNFa (p=0.0016, p=0.0035)). There was an increase in frequency of CD8 expressing GZMB in mice treated with Compound 1+anti-PD-L1 group versus vehicle (p=0.0137). There was an observed increase of the polyfunctionality of the CD8 population (co-expression of IFNg and TNFa) in the Compound 1+/− anti-PD-L1 groups in comparison to the vehicle group (P=0.0008, P=0.0011).

FIG. 6B illustrates that mice treated with Compound 1+anti-PD-L1 increase the total number of CD8 T cell population expressing activation markers in comparison to the vehicle group (KI-67 (p=0.0127), CD69 (p=0.0009), ICOS (p=0.001)).

### DETAILED DESCRIPTION

Unless defined otherwise, all technical and scientific terms used herein have the same meaning as commonly understood by those of ordinary skill in the art to which the invention belongs. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention.

The following definitions are provided to facilitate understanding of certain terms used frequently herein and are not meant to limit the scope of the present disclosure. All references referred to herein are incorporated by reference in their entirety.

As used herein, and unless otherwise specified, the terms “about” and “approximately,” when referring to doses, amounts, or weight percent of ingredients of a composition or a dosage form, mean a dose, amount, or weight percent that is recognized by one of ordinary skill in the art to provide a pharmacological effect equivalent to that obtained from the specified dose, amount, or weight percent. The equivalent dose, amount, or weight percent can be within 30%, 20%, 15%, 10%, 5%, 1%, or less of the specified dose, amount, or weight percent.

“Compound 1” refers to a compound having structure:

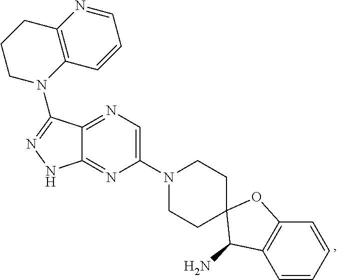 [SMILES: N[C@@H]1c2ccccc2OC12CCN(c1cnc3c(N4CCCc5ncccc54)n[nH]c3n1)CC2]

having the chemical name (R)-1′-(3-(3,4-dihydro-1,5-naphthyridin-1(2H)-yl)-1H-pyrazolo[3,4-b]pyrazin-6-yl)-3H-spiro[benzofuran-2,4′-piperidin]-3-amine.

The term “pharmaceutically acceptable” refers to molecular entities and compositions that do not produce an adverse, allergic or other untoward reaction when administered to an animal, such as, for example, a human, as appropriate.

Compounds of the invention may be in the form of a salt, such as a pharmaceutically acceptable salt. “Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicylic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particular base addition salts are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particular organic non-toxic bases include isopropylamine, diethylamine, ethanolamine, tromethamine, dicyclohexylamine, choline, and caffeine.

In some embodiments, a salt is selected from a hydrochloride, hydrobromide, trifluoroacetate, sulfate, phosphate, acetate, fumarate, maleate, tartrate, lactate, citrate, pyruvate, succinate, oxalate, methanesulfonate, p-toluenesulfonate, bisulfate, benzenesulfonate, ethanesulfonate, malonate, xinafoate, ascorbate, oleate, nicotinate, saccharinate, adipate, formate, glycolate, palmitate, L-lactate, D-lactate, aspartate, malate, L-tartrate, D-tartrate, stearate, furoate (e.g., 2-furoate or 3-furoate), napadisylate (naphthalene-1,5-disulfonate or naphthalene-i-(sulfonic acid)-5-sulfonate), edisylate (ethane-1,2-disulfonate or ethane-1-(sulfonic acid)-2-sulfonate), isothionate (2-hydroxyethylsulfonate), 2-mesitylenesulfonate, 2-naphthalenesulfonate, 2,5-dichlorobenzenesulfonate, D-mandelate, L-mandelate, cinnamate, benzoate, adipate, esylate, malonate, mesitylate (2-mesitylenesulfonate), napsylate (2-naphthalenesulfonate), camsylate (camphor-i0-sulfonate, for example (1S)-(+)-10-camphorsulfonic acid salt), glutamate, glutarate, hippurate (2-(benzoylamino)acetate), orotate, xylate (p-xylene-2-sulfonate), and pamoic (2,2′-dihydroxy-1,1′-dinaphthylmethane-3,3′-dicarboxylate).

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

The terms “PD-L1 binding antagonist,” “PD-L1 inhibitor,” and “PD-L1 blocking antibody” are used interchangeably herein and refer to a molecule that decreases, blocks, inhibits, abrogates, or interferes with signal transduction resulting from the interaction of PD-L1 with either one or more of its binding partners, such as PD-1 and/or 137-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or 137-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MED14736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In a preferred aspect, the PD-L1 binding antagonist is atezolizumab.

The terms “programmed death ligand 1” and “PD-L1” refer herein to native sequence human PD-L1 polypeptide. Native sequence PD-L1 polypeptides are provided under Uniprot Accesion No. Q9NZQ7. For example, the native sequence PD-L1 may have the amino acid sequence as set forth in Uniprot Accesion No. Q9NZQ7-1 (isoform 1). In another example, the native sequence PD-L1 may have the amino acid sequence as set forth in Uniprot Accesion No. Q9NZQ7-2 (isoform 2). In yet another example, the native sequence PD-L1 may have the amino acid sequence as set forth in Uniprot Accesion No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 1; (b) a VL comprising an amino acid sequence comprising having at least 95% sequence identity (e.g., at least 95%, 96%, 97%, 98%, or 99% sequence identity) to, or the sequence of SEQ ID NO: 2; or (c) a VH as in (a) and a VL as in (b).

In one embodiment, the anti-PD-L1 antibody comprises atezolizumab, which comprises:

(a) the heavy chain (VH) amino acid sequence:
(SEQ ID NO: 1)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and
(b) the light chain (VL) amino acid sequence:
(SEQ ID NO: 2)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The term “cancer” refers to a disease caused by an uncontrolled division of abnormal cells in a part of the body. In one instance, the cancer is lung cancer. In another instance, the cancer is non-small cell lung cancer (NSCLC). In one instance, the cancer is head and neck cancer. In another instance, the cancer is head and neck squamous cell carcinoma (HNSCC).

In one instance, the cancer is melanoma. In another instance, the cancer is BRAF Wild Type (WT) melanoma.

As used herein, “treating” comprises effective cancer treatment with an effective amount of a therapeutic agent (e.g., atezolizumab or Compound 1) or combination of therapeutic agents (e.g., atezolizumab and Compound 1). The treatment may be first-line treatment (e.g., the patient may be previously untreated or not have received prior systemic therapy), or second line or later treatment. For example, a patient is successfully “treated” if one or more symptoms associated with a cancer described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a cancer described herein. This delay can be of varying lengths of time, depending on the history of the cancer described herein and/or patient being treated. As is evident to one skilled in the art, a sufficient or significant delay can, in effect, encompass prevention, in that the patient does not develop the cancer.

Herein, an “effective amount” refers to the amount of a therapeutic agent described herein (e.g., atezolizumab and/or Compound 1) that achieves a therapeutic result. In some examples, the effective amount of a therapeutic agent or a combination of therapeutic agents is the amount of the agent or of the combination of agents that achieves a clinical endpoint as provided herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disease. An effective amount can be administered in one or more administrations. An effective amount of drug, compound, pharmaceutical composition, or combination therapy described herein can be an amount sufficient to accomplish therapeutic treatment either directly or indirectly.

“Objective response rate” or “ORR” refers the percentage of patients with a confirmed complete response or partial response on two consecutive occasions³ 4 weeks apart, as determined by the investigator according to RECIST v1.1.

“Duration of response” or “DOR” refers to the time from the first occurrence of a documented objective response to disease progression, as determined by the investigator according to RECIST v1.1, or death from any cause, whichever occurs first.

“Progression free survival” or “PFS” refers to the time from enrollment to the date of the first recorded occurrence of disease progression, as determined by the investigator using RECIST v1.1 or death from any cause, whichever occurs first.

As used herein, “complete response” and “CR” refers to disappearance of all target lesions and (if applicable) normalization of tumor marker level.

As used herein, “partial response” and -PR” refers to persistence of one or more non-target lesions and/or (if applicable) maintenance of tumor marker level above the normal limits. A PR can also refer to³ 30% decrease in sum of diameters of target lesions, in the absence of CR, new lesions, and unequivocal progression in non-target lesions.

An “administration period” or “cycle” refers to a period of time comprising administration of one or more agents described herein (e.g., Compound 1 and atezolizumab) and an optional period of time comprising no administration of one or more of the agents described herein. For example, a cycle can be 21 days in total and include administration of one or more agents described herein (e.g., Compound 1 and atezolizumab) each day of the cycle. In another example, a cycle can be 28 days in total length and include administration of one or more agents described herein (e.g., Compound 1 and atezolizumab) for 21 days and a rest period of seven days. A “rest period” refers to a period of time wherein at least one of the agents described herein (i.e. Compound 1 and atezolizumab) are not administered. In one embodiment, a rest period refers to a period of time wherein none of the agents described herein (i.e. Compound 1 and atezolizumab) are administered. A rest period as provided herein can in some instances include administration of another agent that is not Compound 1 or atezolizumab. In such instances, administration of another agent during a rest period should not interfere or detriment administration of an agent described herein. In one instance, cycle as used herein refers to 21 day cycles without a rest period.

A “dosing regimen” refers to a period of administration of the agents described herein comprising one or more cycles, wherein each cycle can include administration of the agents described herein at different times or in different amounts.
“QD” refers to administration of an agent described herein once daily.
“BID” refers to administration of an agent described herein twice daily.
“Q3W” refers to administration of an agent described herein once every three weeks.
“PO” refers to oral administration of an agent described herein.
“IV” refers to intravenous administration of any agent described herein.

A graded adverse event refers to the severity grading scale as established for by NCI CTCAE. In one embodiment, the adverse event is graded in accordance with the table below.

| Grade | Severity |
|---|---|
| 1 | Mild; asymptomatic or mild symptoms; clinical or diagnostic observations |
| | only; or intervention not indicated |
| 2 | Moderate; minimal, local, or non-invasive intervention indicated; or limiting |
| | age-appropriate instrumental activities of daily living ^{a} |
| 3 | Severe or medically significant, but not immediately life-threatening; |
| | hospitalization or prolongation of hospitalization indicated; disabling; or |
| | limiting self-care activities of daily living ^{b, c} |
| 4 | Life-threatening consequences or urgent intervention indicated ^{d} |
| 5 | Death related to adverse event ^{d} |

The term “patient” refers to a human patient. A patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, y, e, y, and p, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case wherein the final two C-terminal amino acids of the heavy chain are glycine (G446) and lysine (K447). Therefore, the C-terminal lysine (K447), or the C-terminal glycine (G446) and lysine (K447), of the Fc region may or may not be present. Amino acid sequences of heavy chains including an Fc region are denoted herein without the C-terminal lysine (K447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum. supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and Compound 1, or a pharmaceutically acceptable salt thereof. As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every three weeks, the concurrently administered drugs are each administered on day 1 of a three-week cycle.

### Combination Therapies

Provided herein are combination therapies (compositions) comprising Compound 1, or a pharmaceutically acceptable salt thereof, and a PD-L1 binding antagonist.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636.

Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety. In one preferred embodiment, the PD-L1 binding antagonist comprises an anti-PD-L1 antibody. In a more preferred embodiment, the anti-PD-L1 antibody is atezolizumab.

In some instances, the anti-PD-L1 antibody
comprises:
(a) an HVR-H1, HVR-H2, and HVR-H3 sequence of
(SEQ ID NO: 3)
GFTFSDSWIH,
(SEQ ID NO: 4)
AWISPYGGSTYYADSVKG
and
(SEQ ID NO: 5)
RHWPGGFDY,
respectively, and
(b) an HVR-L1, HVR-L2, and HVR-L3 sequence of
(SEQ ID NO: 6)
RASQDVSTAVA,
(SEQ ID NO: 7)
SASFLYS
and
(SEQ ID NO: 8)
QQYLYHPAT,
respectively.
In one embodiment, the anti-PD-LI antibody
comprises:
(a) a heavy chain variable region (VH) comprising
the amino acid sequence:
(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,
and
(b) the light chain variable region (VL)
comprising the amino acid sequence:
(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,
and
(b) the light chain amino acid sequence:
(SEQ ID NO: 2)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, Astrazeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly). In some instances, the anti-PD-L1 antibody is STI-A1014 (Sorrento). STI-A1014 is a human anti-PD-L1 antibody. In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or a glycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is glycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

In one aspect provided herein is a combination therapy comprising Compound 1, or a pharmaceutically acceptable salt thereof, and atezolizumab. In one embodiment, the combination therapies described herein are useful in the treatment of certain types of lung cancer, head and neck cancer, and/or melanoma as described herein. In one embodiment, the lung cancer is NSCLC. In one embodiment, the head and neck cancer is HNSCC. In one embodiment, the melanoma is BRAFT WT melanoma. In one embodiment, Compound 1 is a freebase. In another embodiment, Compound 1 is a pharmaceutically acceptable salt of Compound 1.

In one aspect provided herein is a combination therapy (e.g., a composition) comprising Compound 1, or a pharmaceutically acceptable salt thereof, administered QD on days 1-21 of a first 21-day cycle and an anti-PD-L1 antibody.

In one aspect provided herein is a combination therapy comprising Compound 1, or a pharmaceutically acceptable salt thereof, administered QD on days 1-21 of a first 21-day cycle and atezolizumab administered Q3W on day 1 of the first 21-day cycle.

In one aspect provided herein is a combination therapy comprising Compound 1, or a pharmaceutically acceptable salt thereof, administered QD on days 1-21 of a first 21-day cycle and atezolizumab administered Q2W on day 1 of the first 21-day cycle. In one such embodiment, atezolizumab is administered Q2W at an amount of 840 mg. In one such embodiment, atezolizumab is administered Q2W according to a package insert.

In one embodiment of the combination therapies described herein, Compound 1, or a pharmaceutically acceptable salt thereof, is administered as a fixed dose QD administration.

In one embodiment, the administration is oral (PO), wherein Compound 1, or a pharmaceutically acceptable salt thereof, is formulated as a tablet or capsule. In one such embodiment, Compound 1, or a pharmaceutically acceptable salt thereof, is formulated (and administered) as a film coated tablet. In another such embodiment, Compound 1 is administered as a capsule.

In one embodiment of the combination therapies described herein, Compound 1, or a pharmaceutically acceptable salt thereof, is administered at an amount of about 5 mg-250 mg, 5 mg-200 mg, 5 mg-150 mg, 5 mg-125 mg, 5 mg-120 mg, 5 mg-110 mg, 5 mg 100 mg, 5 mg-80 mg, 5 mg-75 mg, 5 mg-60 mg, 5 mg-50 mg, 5 mg-40 mg, 5 mg-20 mg, 5 mg-10 mg, 20 mg-250 mg, 20 mg-200 mg, 20 mg-150 mg, 20 mg-100 mg, 20 mg-80 mg, 20 mg-75 mg, 20 mg-60 mg, 20 mg-40 mg, 40 mg-250 mg, 40 mg-200 mg, 40 mg-150 mg, 40 mg-100 mg, 40 mg-80 mg, 40 mg-60 mg, 60 mg-250 mg, 60 mg-200 mg, 60 mg-150 mg, 60 mg-100 mg, or 60 mg-80 mg QD.

In one embodiment of the combination therapies described herein, Compound 1, or a pharmaceutically acceptable salt thereof, is administered at an amount of about 5 mg-250 mg, 5 mg-200 mg, 5 mg-150 mg, 5 mg-125 mg, 5 mg-120 mg, 5 mg-110 mg, 5 mg 100 mg, 5 mg-80 mg, 5 mg-75 mg, 5 mg-60 mg, 5 mg-50 mg, 5 mg-40 mg, 5 mg-20 mg, 5 mg-10 mg, 20 mg-250 mg, 20 mg-200 mg, 20 mg-150 mg, 20 mg-100 mg, 20 mg-80 mg, 20 mg-75 mg, 20 mg-60 mg, 20 mg-40 mg, 40 mg-250 mg, 40 mg-200 mg, 40 mg-150 mg, 40 mg-100 mg, 40 mg-80 mg, 40 mg-60 mg, 60 mg-250 mg, 60 mg-200 mg, 60 mg-150 mg, 60 mg-100 mg, or 60 mg-80 mg BID.

In another embodiment, Compound 1, or a pharmaceutically acceptable salt thereof, is administered at an amount of about 5 mg, 10 mg, 20 mg, 40 mg, 50 mg, 60 mg. 80 mg, 100 mg, 120 mg or 150 mg QD. In another embodiment, Compound 1, or a pharmaceutically acceptable salt thereof, is administered at an amount of about 5 mg, 10 mg, 20 mg, 40 mg, 50 mg, 60 mg, 80 mg, 100 mg, 120 mg or 150 mg BID. In another embodiment, Compound 1, or a pharmaceutically acceptable salt thereof, is administered at an amount of about 20 mg, 40 mg, 50 mg, 60 mg, or 80 mg QD. In another embodiment, Compound 1, or a pharmaceutically acceptable salt thereof, is administered at an amount of about 20 mg, 40 mg, 50 mg, 60 mg, or 80 mg BID.

In one embodiment of the combination therapy described herein, the PD-L1 binding antagonist is administered in accordance with a package insert. In a preferred embodiment, the PD-L1 binding antagonist is atezolizumab.

As a general proposition, the therapeutically effective amount of a PD-L1 binding antagonist (e.g., atezolizumab) administered to a human will be in the range of about 0.01 mg/kg to about 50 mg/kg of patient body weight, whether by one or more administrations.

In some exemplary embodiments, the PD-L1 binding antagonist is administered in a dose of about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or every four weeks, for example.

In one instance, a PD-L1 binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-L1 binding antagonist may be administered at a dose of about 1000 mg to about 1400 mg every three weeks (e.g., about 1100 mg to about 1300 mg every three weeks, e.g., about 1150 mg to about 1250 mg every three weeks).

In one preferred embodiment, the combination therapies described herein comprise Compound 1, or a pharmaceutically acceptable salt thereof, as described herein, and atezolizumab, wherein atezolizumab is administered to the patient intravenously at a dose of about 840 mg every two weeks, about 1200 mg every three weeks, or about 1680 mg of every four weeks. In one preferred embodiment, the combination therapies described herein comprise Compound 1, or a pharmaceutically acceptable salt thereof, as described herein, and atezolizumab, wherein atezolizumab is administered to the patient intravenously at a dose of about 1200 mg Q3W.

In one embodiment, the combination therapies described herein are used for treating lung cancer. In one particular embodiment, the combination therapy comprises Compound 1, or a pharmaceutically acceptable salt thereof, and atezolizumab, wherein the combination therapy is for treating lung cancer. In one such embodiment, the lung cancer is non-small cell lung carcinoma (NSCLC). In one such embodiment, the NSCLC is locally advanced or metastatic NSCLC. In another such embodiment, the NSCLC does not have EGFR or ALK alterations.

In one embodiment, the combination therapies described herein are used for treating head and neck cancer. In one such embodiment, the head and neck cancer is head and neck squamous cell carcinoma (HNSCC). In one particular embodiment, the combination therapy comprises Compound 1, or a pharmaceutically acceptable salt thereof, and atezolizumab, wherein the combination therapy is for treating HNSCC. In one such embodiment, the HNSCC is recurrent or metastatic HNSCC involving the oropharynx, oral cavity, larynx, or hypopharynx.

In one embodiment, the combination therapies described herein are used for treating melanoma. In one particular embodiment, the combination therapy comprises Compound 1, or a pharmaceutically acceptable salt thereof, and atezolizumab, wherein the combination therapy is for treating melanoma. In one such embodiment, the melanoma is locally advanced or metastatic (e.g., recurrent or de novo Stage IV) melanoma. In another such embodiment, the melanoma is unresectable locally advanced (e.g., Stage III) cutaneous melanoma. In such embodiments, the melanoma is BRAF WT melanoma.

In still another aspect provided herein is a combination therapy useful in the treatment of lung cancer as described herein, wherein the combination therapy comprises Compound 1, or a pharmaceutically acceptable salt thereof, and atezolizumab, wherein Compound 1, or a pharmaceutically acceptable salt thereof, is administered QD on days 1-21 of a first 21-day cycle and atezolizumab is administered Q3W on day 1 of the first 21-day cycle. In one embodiment, the lung cancer is NSCLC.

In still another aspect provided herein is a combination therapy useful in the treatment of head and neck cancer as described herein, wherein the combination therapy comprises Compound 1, or a pharmaceutically acceptable salt thereof, and atezolizumab, wherein Compound 1 or a pharmaceutically acceptable salt thereof, is administered QD on days 1-21 of a first 21-day cycle and atezolizumab is administered Q3W on day 1 of the first 21-day cycle. In one embodiment, the head and neck cancer is HNSCC.

In still another aspect provided herein is a combination therapy useful in the treatment of melanoma as described herein, wherein the combination therapy comprises Compound 1, or a pharmaceutically acceptable salt thereof, and atezolizumab, wherein Compound 1, or a pharmaceutically acceptable salt thereof, is administered QD on days 1-21 of a first 21-day cycle and atezolizumab is administered Q3W on day 1 of the first 21-day cycle. In one embodiment, the melanoma is BRAFT WT melanoma.

In still another aspect provided herein is a combination therapy useful in the treatment of lung cancer as described herein, wherein the combination therapy comprises Compound 1, or a pharmaceutically acceptable salt thereof, and atezolizumab, wherein Compound 1, or a pharmaceutically acceptable salt thereof, is administered QD at an amount of about 5 mg-100 mg on days 1-21 of a first 21-day cycle and atezolizumab is administered Q3W at an amount of about 1200 mg on day 1 of the first 21-day cycle. In one such embodiment, the lung cancer is NSCLC as described herein.

In still another aspect provided herein is a combination therapy useful in the treatment of head and neck cancer as described herein, wherein the combination therapy comprises Compound 1, or a pharmaceutically acceptable salt thereof, and atezolizumab, wherein Compound 1, or a pharmaceutically acceptable salt thereof, is administered QD at an amount of about 5 mg-100 mg on days 1-21 of a first 21-day cycle and atezolizumab is administered Q3W at an amount of about 1200 mg on day 1 of the first 21-day cycle. In one embodiment, the head and neck cancer is HNSCC.

In still another aspect provided herein is a combination therapy useful in the treatment of melanoma as described herein, wherein the combination therapy comprises Compound 1, or a pharmaceutically acceptable salt thereof, and atezolizumab, wherein Compound 1, or a pharmaceutically acceptable salt thereof, is administered QD at an amount of about 5 mg-100 mg on days 1-21 of a first 21-day cycle and atezolizumab is administered Q3W at an amount of about 1200 mg on day 1 of the first 21-day cycle. In one embodiment, the melanoma is BRAFT WT melanoma.

In still another aspect provided herein is a combination therapy comprising Compound 1, or a pharmaceutically acceptable salt thereof, and atezolizumab for use in the treatment of lung cancer, wherein Compound 1, or a pharmaceutically acceptable salt thereof, is administered QD on days 1-21 of a first 21-day cycle and atezolizumab is administered Q3W on day 1 of the first 21-day cycle. In one embodiment, the lung cancer is NSCLC.

In still another aspect provided herein is a combination therapy comprising Compound 1, or a pharmaceutically acceptable salt thereof, and atezolizumab for use in the treatment of head and neck cancer, wherein Compound 1 or a pharmaceutically acceptable salt thereof, is administered QD on days 1-21 of a first 21-day cycle and atezolizumab is administered Q3W on day 1 of the first 21-day cycle. In one embodiment, the head and neck cancer is HNSCC.

In still another aspect provided herein is a combination therapy comprising Compound 1, or a pharmaceutically acceptable salt thereof, and atezolizumab for use in the treatment of melanoma, wherein Compound 1, or a pharmaceutically acceptable salt thereof, is administered QD on days 1-21 of a first 21-day cycle and atezolizumab is administered Q3W on day 1 of the first 21-day cycle. In one embodiment, the melanoma is BRAFT WT melanoma.

In still another aspect provided herein is a combination therapy comprising Compound 1, or a pharmaceutically acceptable salt thereof, and atezolizumab for use in the treatment of lung cancer, wherein Compound 1, or a pharmaceutically acceptable salt thereof, is administered QD at an amount of about 5 mg-100 mg on days 1-21 of a first 21-day cycle and atezolizumab is administered Q3W at an amount of about 1200 mg on day 1 of the first 21-day cycle. In one such embodiment, the lung cancer is NSCLC as described herein.

In still another aspect provided herein is a combination therapy comprising Compound 1, or a pharmaceutically acceptable salt thereof, and atezolizumab for use in the treatment of head and neck cancer, wherein Compound 1, or a pharmaceutically acceptable salt thereof, is administered QD at an amount of about 5 mg-100 mg on days 1-21 of a first 21-day cycle and atezolizumab is administered Q3W at an amount of about 1200 mg on day 1 of the first 21-day cycle. In one embodiment, the head and neck cancer is HNSCC.

In still another aspect provided herein is a combination therapy comprising Compound 1, or a pharmaceutically acceptable salt thereof, and atezolizumab for use in the treatment of melanoma, wherein Compound 1, or a pharmaceutically acceptable salt thereof, is administered QD at an amount of about 5 mg-100 mg on days 1-21 of a first 21-day cycle and atezolizumab is administered Q3W at an amount of about 1200 mg on day 1 of the first 21-day cycle. In one embodiment, the melanoma is BRAFT WT melanoma.

### Methods of Treating

Also provided herein are methods of treating lung cancer (e.g., NSCLC), head and neck cancer (e.g., HNSCC), or melanoma (e.g., BRAFT WT melanoma) in a patient in need thereof. In one embodiment, the method comprises administering an effective amount of a combination therapy comprising Compound 1, or a pharmaceutically acceptable salt thereof, and a PD-L1 binding antagonist. In another embodiment, the method comprises administering an effective amount of a combination therapy comprising Compound 1, or a pharmaceutically acceptable salt thereof, and atezolizumab.

In one aspect provided herein is a method of treating lung cancer in a patient in need thereof, the method comprising administering an effective amount of a combination therapy comprising Compound 1, or a pharmaceutically acceptable salt thereof, and a PD-L1 binding antagonist. In another aspect provided herein is a method of treating lung cancer in a patient in need thereof, the method comprising administering an effective amount of a combination therapy comprising Compound 1, or a pharmaceutically acceptable salt thereof, and atezolizumab. In one embodiment, the lung cancer is NSCLC.

In one aspect provided herein is a method of treating head and neck cancer in a patient in need thereof, the method comprising administering an effective amount of a combination therapy comprising Compound 1, or a pharmaceutically acceptable salt thereof, and a PD-L1 binding antagonist. In another aspect provided herein is a method of treating head and neck cancer in a patient in need thereof, the method comprising administering an effective amount of a combination therapy comprising Compound 1, or a pharmaceutically acceptable salt thereof, and atezolizumab. In one embodiment, the head and neck cancer is HNSCC.

In one aspect provided herein is a method of treating melanoma in a patient in need thereof, the method comprising administering an effective amount of a combination therapy comprising Compound 1, or a pharmaceutically acceptable salt thereof, and a PD-L1 binding antagonist. In another aspect provided herein is a method of treating melanoma in a patient in need thereof, the method comprising administering an effective amount of a combination therapy comprising Compound 1, or a pharmaceutically acceptable salt thereof, and atezolizumab. In one embodiment, the melanoma is BRAFT WT melanoma.

In one embodiment of the methods described herein, Compound 1, or a pharmaceutically acceptable salt thereof, is administered as a fixed dose QD administration.

In another embodiment, of the methods described herein, Compound 1, or a pharmaceutically acceptable salt thereof, is administered as a fixed dose BID administration. In one embodiment, the administration is oral (PO), wherein Compound 1, or a pharmaceutically acceptable salt thereof, is formulated as a tablet or capsule. In one embodiment, Compound 1, or a pharmaceutically acceptable salt thereof, is administered at an amount of about 5 mg-250 mg, 5 mg-200 mg, 5 mg-150 mg, 5 mg-125 mg, 5 mg-120 mg, 5 mg-110 mg, 5 mg 100 mg, 5 mg-80 mg, 5 mg-75 mg, 5 mg-60 mg, 5 mg-50 mg, 5 mg-40 mg, 5 mg-20 mg, 5 mg-10 mg, 20 mg-250 mg, 20 mg-200 mg, 20 mg-150 mg, 20 mg-100 mg, 20 mg-80 mg, 20 mg-75 mg, 20 mg-60 mg, 20 mg-40 mg, 40 mg-250 mg, 40 mg-200 mg, 40 mg-150 mg, 40 mg-100 mg, 40 mg-80 mg, 40 mg-60 mg, 60 mg-250 mg, 60 mg-200 mg, 60 mg-150 mg, 60 mg-100 mg, or 60 mg-80 mg QD.

In another embodiment, Compound 1, or a pharmaceutically acceptable salt thereof, is formulated as a tablet and administered at an amount of about 5 mg-100 mg QD. In one embodiment, Compound 1, or a pharmaceutically acceptable salt thereof, is administered QD at an amount of about 10 mg. In one embodiment, Compound 1, or a pharmaceutically acceptable salt thereof, is administered QD at an amount of about 20 mg. In one embodiment, Compound 1, or a pharmaceutically acceptable salt thereof, is administered QD at an amount of about 40 mg. In one embodiment, Compound 1, or a pharmaceutically acceptable salt thereof, is administered QD at an amount of about 60 mg. In one embodiment, Compound 1, or a pharmaceutically acceptable salt thereof, is administered QD at an amount of about 80 mg.

In one embodiment of the methods described herein, Compound 1, or a pharmaceutically acceptable salt thereof, is administered at an amount of about 5 mg-250 mg, 5 mg-200 mg, 5 mg-150 mg, 5 mg-125 mg, 5 mg-120 mg, 5 mg-110 mg, 5 mg 100 mg, 5 mg-80 mg, 5 mg-75 mg, 5 mg-60 mg, 5 mg-50 mg, 5 mg-40 mg, 5 mg-20 mg, 5 mg-10 mg, 20 mg-250 mg, 20 mg-200 mg, 20 mg-150 mg, 20 mg-100 mg, 20 mg-80 mg, 20 mg-75 mg, 20 mg-60 mg, 20 mg-40 mg, 40 mg-250 mg, 40 mg-200 mg, 40 mg-150 mg, 40 mg-100 mg, 40 mg-80 mg, 40 mg-60 mg, 60 mg-250 mg, 60 mg-200 mg, 60 mg-150 mg, 60 mg-100 mg, or 60 mg-80 mg BID.

In one embodiment of the methods described herein, atezolizumab is administered in a dose of about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or every four weeks, for example.

In one instance, atezolizumab is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, atezolizumab may be administered at a dose of about 1000 mg to about 1400 mg every three weeks (e.g., about 1100 mg to about 1300 mg every three weeks, e.g., about 1150 mg to about 1250 mg every three weeks).

In one preferred embodiment of the methods described herein, Compound 1, or a pharmaceutically acceptable salt thereof, is administered as described herein and atezolizumab is administered to the patient intravenously at a dose of about 1200 mg Q3W.

In one embodiment, the methods described herein further include a run-in period.

The run-in period can be about 1-14 days in length. In one such embodiment, the run-in period comprises 14 days. In another such embodiment, the run-in period is 14 days and Compound 1, or a pharmaceutically acceptable salt thereof, and atezolizumab are administered on day 1 of the run-in period. In one such embodiment, Compound 1, or a pharmaceutically acceptable salt thereof, is administered alone on day 8 of the run-in period and QD thereafter.

The methods provided herein can include administration of a combination therapy described herein as part of a dosing regimen. In such one embodiment, the dosing regimen comprises one or more cycles. In another embodiment, the dosing regimen comprises at least two cycles. In another aspect provided herein is the dosing regimen comprises 2, 3, 4, 5, 6, 8, 10, 12, 16, 18, 20, 24, 30, 36, 42, 48, 54, 60, 66, or 72 cycles. In still another embodiment, dosing regimen comprises about 2-72, 2-66, 2-60, 2-54, 2-48, 2-42, 2-36, 2-30, 2-24, 2-18, 2-12, or 2-6 cycles. In one embodiment, the dosing regimen includes administration of a combination therapy as described herein in any number of cycles until the desired response (e.g., PFS, OS, ORR, and/or DOR) reaches a desired outcome (e.g., increase in PFS, OS, ORR, and/or DOR compared to a control described herein). In another embodiment, the dosing regimen includes administration of a combination therapy as described herein in any number of cycles until toxicity develops or the patient otherwise experiences one or more adverse events (AEs) that prevents further administration. In still another embodiment, the dosing regimen includes administration of a combination therapy as described herein in any number of cycles until disease progression.

In one embodiment of the methods described herein, a patient is administered a total of 1 to 50 doses of atezolizumab, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In one preferred embodiment, the doses are administered intravenously.

In certain embodiments, the therapeutic agents of the combination therapies described herein (e.g., Compound 1, or a pharmaceutically acceptable salt thereof, and atezolizumab) may be administered in any suitable manner known in the art. For example, atezolizumab may be administered sequentially (on different days) or concurrently (on the same day or during the same treatment cycle) as Compound 1, or a pharmaceutically acceptable salt thereof. In one embodiment, atezolizumab is administered after administration of Compound 1, or a pharmaceutically acceptable salt thereof. In some instances, atezolizumab is administered after administration of Compound 1, or a pharmaceutically acceptable salt thereof, may be administered on the same day. In one embodiment, atezolizumab may be administered after administration of Compound 1, or a pharmaceutically acceptable salt thereof, on the same day. For example, Compound 1, or a pharmaceutically acceptable salt thereof, can be administered on day 1 of each cycle prior to administration of atezolizumab on day 1 of each cycle, wherein Compound 1, or a pharmaceutically acceptable salt thereof, is then administered QD for the next 20 days of the 21-day cycle.

In a preferred embodiment, atezolizumab is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

In one embodiment, atezolizumab is administered in accordance with the Table below:

| First Infusion | Subsequent Infusions |
|---|---|
| No premedication is permitted prior to the | If the patient experienced an infusion-related |
| atezolizumab infusion. | reaction with any previous infusion, |
| Vital signs (pulse rate, respiratory rate, blood | premedication with antihistamines, |
| pressure, and temperature) should be measured | antipyretics, and/or analgesics may be |
| within 60 minutes prior to the infusion. | administered for subsequent doses at the |
| Atezolizumab should be infused over 60 (±15) | discretion of the investigator. |
| minutes. | Vital signs should be measured within 60 |
| If clinically indicated, vital signs should be | minutes prior to the infusion. |
| measured every 15 (±5) minutes during the | Atezolizumab should be infused over 30 (±10) |
| infusion and at 30 (±10) minutes after the | minutes if the previous infusion was |
| infusion. | tolerated without an infusion-related reaction, |
| Patients should be informed about the | or 60 (±15) minutes if the patient |
| possibility of delayed post-infusion symptoms | experienced an infusion-related reaction with |
| and instructed to contact their study physician if | the previous infusion. |
| they develop such symptoms. | If the patient experienced an infusion-related |
| | reaction with the previous infusion or if |
| | clinically indicated, vital signs should be |
| | measured during the infusion and at 30 (±10) |
| | minutes after the infusion. |

Also provided herein are methods for treating lung cancer (e.g., NSCLC), head and neck cancer (e.g., HNSCC), or melanoma (e.g., BRAFT WT melanoma) in a patient in need thereof, wherein the method comprises administering to the patient a treatment regimen comprising an effective amount of Compound 1, or a pharmaceutically acceptable salt thereof, and a PD-L1 binding antagonist (e.g., atezolizumab). In another embodiment of such methods, Compound 1, or a pharmaceutically acceptable salt thereof, is administered QD as described herein and in an amount as described herein (e.g., 5 mg-100 mg). In another embodiment of such methods, atezolizumab is administered Q3W as described herein and in an amount as described herein (e.g., 1200 mg). In another such embodiment, Compound 1, or a pharmaceutically acceptable salt thereof, and atezolizumab are administered as a component of a run-in period as described herein prior to the start of the treatment regimen. In such methods, Compound 1, or a pharmaceutically acceptable salt thereof, and atezolizumab can be administered as described herein.

Further provided herein are methods for treating lung cancer in a patient in need thereof, wherein the method comprises administering to the patient a treatment regimen comprising an effective amount of Compound 1, or a pharmaceutically acceptable salt thereof, and a PD-L1 binding antagonist (e.g., atezolizumab). In another embodiment of such methods, Compound 1, or a pharmaceutically acceptable salt thereof, is administered QD as described herein and in an amount as described herein (e.g., 5 mg-100 mg). In another embodiment of such methods, atezolizumab is administered Q3W as described herein and in an amount as described herein (e.g., 1200 mg). In another such embodiment, Compound 1, or a pharmaceutically acceptable salt thereof, and atezolizumab are administered as a component of a run-in period as described herein prior to the start of the treatment regimen. In such methods, Compound 1, or a pharmaceutically acceptable salt thereof, and atezolizumab can be administered as described herein. In one such embodiment, the lung cancer is NSCLC as described herein.

Still further provided herein are methods for treating head and neck cancer as described herein in a patient in need thereof, wherein the method comprises administering to the patient a treatment regimen comprising an effective amount of Compound 1, or a pharmaceutically acceptable salt thereof, and a PD-L1 binding antagonist (e.g., atezolizumab). In another embodiment of such methods, Compound 1, or a pharmaceutically acceptable salt thereof, is administered QD as described herein and in an amount as described herein (e.g., 5 mg-100 mg). In another embodiment of such methods, atezolizumab is administered Q3W as described herein and in an amount as described herein (e.g., 1200 mg).

In another such embodiment, Compound 1, or a pharmaceutically acceptable salt thereof, and atezolizumab are administered as a component of a run-in period as described herein prior to the start of the treatment regimen. In such methods, Compound 1, or a pharmaceutically acceptable salt thereof, and atezolizumab can be administered as described herein. In one embodiment, the head and neck cancer is HNSCC as described herein.

Also provided herein are methods for treating melanoma as described herein in a patient in need thereof, wherein the method comprises administering to the patient a treatment regimen comprising an effective amount of Compound 1, or a pharmaceutically acceptable salt thereof, and a PD-L1 binding antagonist (e.g., atezolizumab). In another embodiment of such methods, Compound 1, or a pharmaceutically acceptable salt thereof, is administered QD as described herein and in an amount as described herein (e.g., 5 mg-100 mg). In another embodiment of such methods, atezolizumab is administered Q3W as described herein and in an amount as described herein (e.g., 1200 mg). In another such embodiment, Compound 1, or a pharmaceutically acceptable salt thereof, and atezolizumab are administered as a component of a run-in period as described herein prior to the start of the treatment regimen. In such methods, Compound 1, or a pharmaceutically acceptable salt thereof, and atezolizumab can be administered as described herein. In one embodiment, the melanoma is BRAFT WT melanoma as described herein.

In some instances, a treatment regimen as described herein includes administration of one or more additional therapies wherein the additional therapy is one or more side-effect limiting agents (e.g., agents intended to lessen the occurrence and/or severity of side effects of treatment, such as anti-nausea agents, acid reducers, a corticosteroid (e.g., prednisone or an equivalent, e.g., at a dose of 1-2 mg/kg/day), hormone replacement medicine(s), and the like).

In some embodiments of the methods described herein, a patient is evaluated for PD-L1 positivity or PD-L1 status. In one embodiment, PD-L1 positivity or PD-L1 status is assess by IHC-based testing. In one embodiment, a patient is PD-L1 positive. In one embodiment, a patient has NSCLC assessed with a PD-L1 high status. In another embodiment, a patient has NSCLC assessed with a PD-L1 low status. In one such embodiment, the patient has NSCLC with EGFR and ALK wild-type status.

The expression of PD-L1 may be assessed in a patient treated according to any of the methods and compositions for use described herein. The methods and compositions for use may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. Patent Application Publication Nos. 2018/0030138 and 2018/0037655. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 10 to less than 5% of the tumor cells in the tumor sample, in 5bl or more of the tumor cells in the tumor sample, in from 5 to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 10% to less than 5% of the tumor sample, more than 5% of the tumor sample, from 50 to less than 0% of the tumor sample, or more than 100 of the tumor sample.

In some examples, tumor samples may be scored for PD-L1 positivity in tumor-infiltrating immune cells and/or in tumor cells according to the criteria for diagnostic assessment shown in Table A and/or Table B, respectively.

TABLE A
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any intensity in tumor-infiltrating | |
| immune cells covering <1% of tumor area occupied by tumor cells, associated | |
| intratumoral stroma, and contiguous peri-tumoral desmoplastic stroma | |
| Presence of discernible PD-L1 staining of any intensity in tumor-infiltrating | IC1 |
| immune cells covering ≥1% to <5% of tumor area occupied by tumor cells, | |
| associated intratumoral stroma, and contiguous peri-tumoral desmoplastic | |
| stroma | |
| Presence of discernible PD-L1 staining of any intensity in tumor-infiltrating | IC2 |
| immune cells covering ≥5% to <10% of tumor area occupied by tumor cells, | |
| associated intratumoral stroma, and contiguous peri-tumoral desmoplastic | |
| stroma | |
| Presence of discernible PD-L1 staining of any intensity in tumor-infiltrating | IC3 |
| immune cells covering ≥10% of tumor area occupied by tumor cells, | |
| associated intratumoral stroma, and contiguous peri-tumoral desmoplastic | |
| stroma | |

TABLE B
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any intensity in <1% of tumor cells | |
| Presence of discernible PD-L1 staining of any intensity in ≥1% to <5% of | TC1 |
| tumor cells | |
| Presence of discernible PD-L1 staining of any intensity in ≥5% to <50% of | TC2 |
| tumor cells | |
| Presence of discernible PD-L1 staining of any intensity in ≥50% of tumor cells | TC3 |

Also provided herein are methods of inhibiting tumor growth or producing tumor regression in a patient described herein by administering a combination therapy described herein. In one embodiment provided herein is a method of inhibiting tumor growth in a patient in need thereof by administering a combination therapy comprising administering Compound 1, or a pharmaceutically acceptable salt thereof, and atezolizumab in one or more 21-day cycles as described herein.

In one embodiment provided herein is a method of producing or improving tumor regression in a patient having a lung cancer (e.g., NSCLC), head and neck cancer (e.g., HNSCC), or melanoma (e.g., BRAFT WT melanoma) as described herein by administering a combination therapy comprising administering Compound 1, or a pharmaceutically acceptable salt thereof, and atezolizumab in one or more 21-day cycles as described herein.

Further provided herein is the use of a combination therapy (composition) described herein comprising Compound 1, or a pharmaceutically acceptable salt thereof, and atezolizumab for the treatment of lung cancer (e.g., NSCLC), head and neck cancer (e.g., HNSCC), or melanoma (e.g., BRAFT WT melanoma) as described herein.

In one embodiment, is a use (U1) of a combination therapy (composition) described herein comprising Compound 1, or a pharmaceutically acceptable salt thereof, and atezolizumab for the treatment of lung cancer as described herein. In one such embodiment, the lung cancer is NSCLC. In some embodiments, the NSCLC is locally advanced NSCLC or metastatic NSCLC as described herein.

In one embodiment, is a use (U2) of a combination therapy (composition) described herein comprising Compound 1, or a pharmaceutically acceptable salt thereof, and atezolizumab for the treatment of head and neck cancer as described herein. In one such embodiment, the head and neck cancer is HNSCC as described herein.

In one embodiment, is a use (U3) of a combination therapy (composition) described herein comprising Compound 1, or a pharmaceutically acceptable salt thereof, and atezolizumab for the treatment of melanoma as described herein. In one embodiment, the melanoma is BRAFT WT melanoma as described herein.

Further provided herein is a use (U4) of a combination therapy described herein comprising Compound 1, or a pharmaceutically acceptable salt thereof, and atezolizumab for the treatment of lung cancer, head and neck cancer, or melanoma as described herein comprising a dosing regimen comprising: (i) administering Compound 1, or a pharmaceutically acceptable salt thereof, QD on days 1-21 of a first 21-day cycle; and (ii) administering atezolizumab Q3W on day 1 of the first 21-day cycle. In one such embodiment, the use comprises treatment of lung cancer. In one such embodiment, the lung cancer is NSCLC as described herein. In another such embodiment, the use comprises treatment of head and neck cancer. In one such embodiment, the head and neck cancer is HNSCC as described herein. In another such embodiment, the use comprises treatment of melanoma as described herein. In one such embodiment, the melanoma is BRAFT WT melanoma as described herein.

Further provided herein is the use (U5) of a combination therapy described herein comprising Compound 1, or a pharmaceutically acceptable salt thereof, and atezolizumab for the treatment of lung cancer, head and neck cancer, or melanoma as described herein comprising a dosing regimen comprising: (i) administering about 5-100 mg Compound 1, or a pharmaceutically acceptable salt thereof, QD on days 1-21 of a first 21-day cycle; and (ii) administering about 1200 mg atezolizumab Q3W on day 1 of the first 21-day cycle. In one such embodiment, the dosing regimen includes two or more cycles as described herein. In one such embodiment, the use comprises treatment of lung cancer. In one such embodiment, the lung cancer is NSCLC as described herein. In another such embodiment, the use comprises treatment of head and neck cancer. In one such embodiment, the head and neck cancer is HNSCC as described herein. In another such embodiment, the use comprises treatment of melanoma as described herein. In one such embodiment, the melanoma is BRAFT WT melanoma as described herein.

Further provided herein is the use (U6) of a combination therapy described herein comprising Compound 1, or a pharmaceutically acceptable salt thereof, and atezolizumab for the manufacture of a medicament for the treatment of lung cancer, head and neck cancer, or melanoma as described herein. In one such embodiment, the use comprises treatment of lung cancer. In one such embodiment, the lung cancer is NSCLC as described herein. In another such embodiment, the use comprises treatment of head and neck cancer. In one such embodiment, the head and neck cancer is HNSCC as described herein. In another such embodiment, the use comprises treatment of melanoma as described herein. In one such embodiment, the melanoma is BRAFT WT melanoma as described herein.

Further provided herein is the use (U7) of a combination therapy described herein comprising Compound 1, or a pharmaceutically acceptable salt thereof, and atezolizumab for the manufacture of a medicament for the treatment of lung cancer, head and neck cancer, or melanoma as described herein comprising a dosing regimen comprising: (i) administering Compound 1, or a pharmaceutically acceptable salt thereof, QD on days 1-21 of a first 21-day cycle; and (ii) administering atezolizumab Q3W on day 1 of the first 21-day cycle. In one such embodiment, the use comprises treatment of lung cancer. In one such embodiment, the lung cancer is NSCLC as described herein. In another such embodiment, the use comprises treatment of head and neck cancer. In one such embodiment, the head and neck cancer is HNSCC as described herein. In another such embodiment, the use comprises treatment of melanoma as described herein. In one such embodiment, the melanoma is BRAFT WT melanoma as described herein.

Further provided herein is the use (U7) of a combination therapy described herein comprising Compound 1, or a pharmaceutically acceptable salt thereof, and atezolizumab for the manufacture of a medicament for the treatment of lung cancer, head and neck cancer, or melanoma as described herein comprising a dosing regimen comprising: (i) administering about 5-100 mg Compound 1, or a pharmaceutically acceptable salt thereof, QD on days 1-21 of a first 21-day cycle; and (ii) administering about 1200 mg atezolizumab Q3W on day 1 of the first 21-day cycle. In one such embodiment, the dosing regimen includes two or more cycles as described herein. In one such embodiment, the use comprises treatment of lung cancer. In one such embodiment, the lung cancer is NSCLC as described herein. In another such embodiment, the use comprises treatment of head and neck cancer. In one such embodiment, the head and neck cancer is HNSCC as described herein. In another such embodiment, the use comprises treatment of melanoma as described herein. In one such embodiment, the melanoma is BRAFT WT melanoma as described herein.

In one embodiment, a combination therapy (composition) described herein comprises Compound 1, or a pharmaceutically acceptable salt thereof, and atezolizumab for use (U1) in the treatment of lung cancer as described herein. In one such embodiment, the lung cancer is NSCLC. In some embodiments, the NSCLC is locally advanced NSCLC or metastatic NSCLC as described herein.

In one embodiment, a combination therapy (composition) described herein comprises Compound 1, or a pharmaceutically acceptable salt thereof, and atezolizumab for use (U2) in the treatment of head and neck cancer as described herein. In one such embodiment, the head and neck cancer is HNSCC as described herein.

In one embodiment, a combination therapy (composition) described herein comprises Compound 1, or a pharmaceutically acceptable salt thereof, and atezolizumab for use (U3) in the treatment of melanoma as described herein. In one such embodiment, the melanoma is BRAFT WT melanoma as described herein.

Further provided herein a combination therapy described herein comprising Compound 1, or a pharmaceutically acceptable salt thereof, and atezolizumab for use (U4) in the treatment of lung cancer, head and neck cancer, or melanoma as described herein comprising a dosing regimen comprising: (i) administering Compound 1, or a pharmaceutically acceptable salt thereof, QD on days 1-21 of a first 21-day cycle; and (ii) administering atezolizumab Q3W on day 1 of the first 21-day cycle. In one such embodiment, the treatment comprises treatment of lung cancer. In one such embodiment, the lung cancer is NSCLC as described herein. In another such embodiment, the treatment comprises treatment of head and neck cancer. In one such embodiment, the head and neck cancer is HNSCC as described herein. In another such embodiment, the treatment comprises treatment of melanoma as described herein. In one such embodiment, the melanoma is BRAFT WT melanoma as described herein.

Further provided herein a combination therapy described herein comprising Compound 1, or a pharmaceutically acceptable salt thereof, and atezolizumab for use (U5) in the treatment of lung cancer, head and neck cancer, or melanoma as described herein comprising a dosing regimen comprising: (i) administering about 5-100 mg Compound 1, or a pharmaceutically acceptable salt thereof, QD on days 1-21 of a first 21-day cycle; and (ii) administering about 1200 mg atezolizumab Q3W on day 1 of the first 21-day cycle. In one such embodiment, the dosing regimen includes two or more cycles as described herein. In one such embodiment, the treatment comprises treatment of lung cancer. In one such embodiment, the lung cancer is NSCLC as described herein. In another such embodiment, the treatment comprises treatment of head and neck cancer. In one such embodiment, the head and neck cancer is HNSCC as described herein. In another such embodiment, the treatment comprises treatment of melanoma as described herein. In one such embodiment, the melanoma is BRAFT WT melanoma as described herein.

Further provided herein a combination therapy described herein comprising Compound 1, or a pharmaceutically acceptable salt thereof, and atezolizumab for use (U6) in the manufacture of a medicament for the treatment of lung cancer, head and neck cancer, or melanoma as described herein. In one such embodiment, the treatment comprises treatment of lung cancer. In one such embodiment, the lung cancer is NSCLC as described herein. In another such embodiment, the treatment comprises treatment of head and neck cancer. In one such embodiment, the head and neck cancer is HNSCC as described herein. In another such embodiment, the treatment comprises treatment of melanoma as described herein. In one such embodiment, the melanoma is BRAFT WT melanoma as described herein.

Further provided herein a combination therapy described herein comprising Compound 1, or a pharmaceutically acceptable salt thereof, and atezolizumab for use (U7) in the manufacture of a medicament for the treatment of lung cancer, head and neck cancer, or melanoma as described herein comprising a dosing regimen comprising: (i) administering Compound 1, or a pharmaceutically acceptable salt thereof, QD on days 1-21 of a first 21-day cycle; and (ii) administering atezolizumab Q3W on day 1 of the first 21-day cycle. In one such embodiment, the treatment comprises treatment of lung cancer. In one such embodiment, the lung cancer is NSCLC as described herein. In another such embodiment, the treatment comprises treatment of head and neck cancer. In one such embodiment, the head and neck cancer is HNSCC as described herein. In another such embodiment, the treatment comprises treatment of melanoma as described herein. In one such embodiment, the melanoma is BRAFT WT melanoma as described herein.

Further provided herein a combination therapy described herein comprising Compound 1, or a pharmaceutically acceptable salt thereof, and atezolizumab for use (U7) in the manufacture of a medicament for the treatment of lung cancer, head and neck cancer, or melanoma as described herein comprising a dosing regimen comprising: (i) administering about 5-100 mg Compound 1, or a pharmaceutically acceptable salt thereof, QD on days 1-21 of a first 21-day cycle; and (ii) administering about 1200 mg atezolizumab Q3W on day 1 of the first 21-day cycle. In one such embodiment, the dosing regimen includes two or more cycles as described herein. In one such embodiment, the treatment comprises treatment of lung cancer. In one such embodiment, the lung cancer is NSCLC as described herein. In another such embodiment, the treatment comprises treatment of head and neck cancer. In one such embodiment, the head and neck cancer is HNSCC as described herein. In another such embodiment, the treatment comprises treatment of melanoma as described herein. In one such embodiment, the melanoma is BRAFT WT melanoma as described herein.

The development of combination treatments poses challenges including, for example, the selection of agents for combination therapy that may lead to improved efficacy while maintaining acceptable toxicity. One particular challenge is the need to distinguish the incremental toxicity of the combination. In one embodiment of the methods described herein the combination therapy described herein (e.g., Compound 1, or a pharmaceutically acceptable salt thereof, and atezolizumab) is administered in a dosing regimen comprising a staggered dosing schedule. In one such embodiment, the patient has a reduced number or grade of adverse events (AEs) comparable to a control (e.g., SOC therapy, treatment with one agent described herein (e.g., Compound 1, or a pharmaceutically acceptable salt thereof, or atezolizumab) alone).

It is generally understood that when an adverse event occurs, four options exist: (1) continue treatment as-is with optional concomitant therapy; (2) adjust the dose of one or more agents in the dosing regimen; (3) suspend administration of one or more agents in the dosing regimen; or (4) discontinue administration of one or more agents in the dosing regimen. In one embodiment, the amount of Compound 1, or a pharmaceutically acceptable salt thereof, is not modified. In another embodiment, the amount of atezolizumab administered is not modified. In one embodiment, wherein the administration of atezolizumab is interrupted, the next administration of Compound 1, or a pharmaceutically acceptable salt thereof, occurs on the same day as administration of atezolizumab is resumed.

Patients described herein can experience one or more adverse events (AEs) such as those described herein. In one embodiment, a patient described herein experiences peripheral edema, myelosuppression (e.g., Thrombocytopenia and/or anemia), gastrointestinal toxicity (e.g., Diarrhea, nausea, and/or vomiting), rash, elevation of hepatic transaminase, cardiomyopathy, ocular toxicity, or phototoxicity. In another embodiment, a patient described herein experiences one or more of infusion related reactions (IRRs) and immune-mediated hepatitis, pneumonitis, colitis, pancreatitis, diabetes mellitus, hypothyroidism, hyperthyroidism, adrenal insufficiency, hypophysitis, Guillain-Barre syndrome, myasthenic syndrome or myasthenia gravis, meningoencephalitis, myocarditis, nephritis, and myositis.

Immune-mediated reactions may involve any organ system and may lead to hemophagocytic lymphohistiocytosis (HLH) and macrophage activation syndrome (MAS). In one such embodiment, a patient does not experience one or more of cardiac, pulmonary, renal, thromboembolic, or ophthalmic toxicity.

In some embodiments, a patient described herein can also be administered one or more concomitant therapies including: (a) Oral contraceptives; (b) Hormone-replacement therapy; (c) Prophylactic or therapeutic anticoagulation therapy (such as warfarin at a stable dose or low-molecular-weight heparin); Vaccinations (such as influenza, COVID-19), however, Live, attenuated vaccines are not permitted; (d) Megestrol acetate administered as an appetite stimulant; (e) Mineralocorticoids (e.g., fludrocortisone); (f) Inhaled or low-dose corticosteroids administered for chronic obstructive pulmonary disease or asthma; (g) Low-dose corticosteroids administered for orthostatic hypotension or adrenocortical insufficiency; (h) Palliative radiotherapy (e.g., treatment of known bony metastases or symptomatic relief of pain); (i) Local therapy (e.g., surgery, stereotactic radiosurgery, radiotherapy, radiofrequency ablation); (j) Premedication with antihistamines, antipyretics, and/or analgesics; (e.g., acetaminophen, ibuprofen, diphenhydramine, and/or H₂-receptor antagonists (e.g., famotidine, cimetidine).

Patients described herein may not, in certain embodiments, take concomitant therapies including:(a) Strong/moderate CYP3A4 inhibitors (e.g., atazanavir, ritonavir, indinavir, nelfinavir, saquinavir, clarithromycin, telithromycin, erythromycin, troleandomycin, fluconazole, itraconazole, ketoconazole, voriconazole, posaconazole, aprepitant, conivaptan, fluvoxamine, diltiazem, nefazodone, mibefradil, verapamil, and grapefruit juice or grapefruit supplements); (b) Strong/moderate CYP3A4 inducers (e.g., rifampin, carbamazepine, phenytoin, oxcarbazepine, phenobarbital, efavirenz, nevirapine, etravirine, modafinil, hyperforin (St. John's Wort), and cyproterone); (c) chemotherapy; (d) hormonal therapy; (e) immunotherapy; (f) radiotherapy; (g) herbal therapy); (h) Live, attenuated vaccines; Systemic immunostimulatory agents (e.g., interferons and IL-2); (i) Proton-pump inhibitors; (j) Cannabidiol products; or (k) H₂ blockers and antacids.

In one embodiment of the methods described herein, a patient described herein has a locally advanced or metastatic solid tumor (e.g., NSCLC, HNSCC, melanoma as described herein) that has either: (a) progressed after at least one available standard therapy, or (b) for which approved standard therapy has proven to be ineffective or intolerable, or is considered inappropriate.

In one embodiment of the methods described herein, the patient has lung cancer and has not received prior systemic therapy. In one such embodiment, the lung cancer is locally advanced or metastatic lung cancer as described herein.

In one embodiment of the methods described herein, the patient has NSCLC and has not received prior systemic therapy. In one such embodiment, the NSCLC is locally advanced or metastatic NSCLC as described herein.

In another embodiment of the methods described herein, the patient has NSCLC as described herein and the absence of EGFR and/or ALK alterations. In one such embodiment, a patient sample (e.g., biopsy of tumor tissue) is tested for the absence or presence of EGFR and/or ALK alterations. In another such embodiment, the patient is PD-L1 positive. In such embodiments, a patient is determined as PD-L1 positive by performing IHC analysis as described herein.

In one embodiment of the methods described herein, a patient described herein has histologically confirmed recurrent or metastatic head and neck cancer. In one such embodiment, the head and neck cancer involves the oropharynx, oral cavity, larynx, or hypopharynx. In one such embodiment, the head and neck cancer involves the oropharynx, oral cavity, larynx, or hypopharynx that is considered not amenable to curative therapy. In another embodiment of the methods described herein, a patient described herein has head and neck cancer as described herein and has not received prior systemic therapy for the treatment of head and neck cancer. In another embodiment of the methods described herein, a patient described herein has head and neck cancer and has been undergone testing for presence/absence of local human papillomavirus (HPV) for oropharyngeal carcinoma.

In one embodiment of the methods described herein, a patient described herein has histologically confirmed recurrent or metastatic HNSCC. In one such embodiment, the HNSCC involves the oropharynx, oral cavity, larynx, or hypopharynx. In one such embodiment, the HNSCC involves the oropharynx, oral cavity, larynx, or hypopharynx that is considered not amenable to curative therapy. In another embodiment of the methods described herein, a patient described herein has HNSCC as described herein and has not received prior systemic therapy for the treatment of HNSCC. In another embodiment of the methods described herein, a patient described herein has HNSCC and has been undergone testing for presence/absence of local human papillomavirus (HPV) for oropharyngeal carcinoma.

In one embodiment of the methods described herein, a patient described herein has melanoma as described herein and has progressed disease on or after prior treatment comprising an anti-PD-1 or anti-PD-L1 therapy.

In one embodiment of the methods described herein, a patient described herein has BRAFT WT melanoma as described herein and has progressed disease on or after prior treatment comprising an anti-PD-1 or anti-PD-L1 therapy.

In one embodiment, a patient described herein does not have known and untreated, or active central nervous system (CNS) metastases (progressing or requiring anticonvulsants or corticosteroids for symptomatic control). A patient may be treated using the methods described herein wherein such patients have a history of treated CNS metastases wherein such a patient has: (1) measurable or evaluable disease outside the CNS; (2) no history of intracranial hemorrhage or spinal cord hemorrhage; (3) no ongoing requirement for corticosteroids as therapy for CNS metastases, with corticosteroids discontinued for two weeks prior to administration of a combination therapy as described herein and no ongoing symptoms attributed to CNS metastases; (4) no stereotactic radiation within seven days or whole-brain radiation within 14 days prior to day 1 of cycle 1 as described herein; and (5) no evidence of interim progression between the completion of CNS-directed therapy and the screening radiographic study.

In one embodiment of the methods described herein, a patient described herein does not have leptomeningeal disease or carcinomatous meningitis. In another embodiment, a patient described herein does not have uncontrolled hypertension. In another embodiment, a patient described herein does not have an active tuberculosis infection. In another embodiment, a patient described herein is not being actively treated for HBV.

In another embodiment of the methods described herein, a patient described herein does not have ophthalmic disease including retinal vein occlusion (RVO), central serous retinopathy (CSR), predisposing factors to RVO or CSR, or retinopathy at the screening ophthalmic examination.

In one embodiment of the methods described herein, a patient described herein has not received a major surgical procedure, other than for diagnosis, within 4 weeks prior to initiation of treatment as described herein.

In another embodiment of the methods described herein, a patient described herein does not have active or history of autoimmune disease or immune deficiency, including, but not limited to, myasthenia gravis, myositis, autoimmune hepatitis, systemic lupus erythematosus, rheumatoid arthritis, inflammatory bowel disease, anti-phospholipid antibody syndrome, Wegener granulomatosis, Sjogren syndrome, Guillain-Barr6 syndrome, or multiple sclerosis. In some embodiments, a patient with such an autoimmune disease can be treating according to the methods described herein if.
such a patient has a history of autoimmune-related hypothyroidism and is being treated with a thyroid-replacement hormone;
such a patient has controlled Type 1 diabetes mellitus and is being treated with an insulin regimen;
such a patient has eczema, psoriasis, lichen simplex chronicus, or vitiligo with dermatologic manifestations only (e.g., patients with psoriatic arthritis are excluded) and (i) the rash covers <10% of body surface area of the patient, (ii) treatment uses only low-potency topical corticosteroids and (iii) there has been no occurrence of acute exacerbations of the underlying condition requiring psoralen plus ultraviolet A radiation, methotrexate, retinoids, biologic agents, oral calcineurin inhibitors, or high-potency or oral corticosteroids within the previous twelve months.

In another embodiment of the methods described herein, a patient does not have a history of idiopathic pulmonary fibrosis, organizing pneumonia (e.g., bronchiolitis obliterans), drug-induced pneumonitis, or idiopathic pneumonitis, or evidence of active pneumonitis on screening chest computed tomography scan.

In another embodiment, a patient described herein has not received treatment with chemotherapy, immunotherapy, or biologic therapy as anti-cancer therapy within three weeks prior to administration of a combination therapy described herein, or endocrine therapy within two weeks prior to administration of a combination therapy described herein, except for hormonal therapy with gonadotropin-releasing hormone (GnRH) agonists or antagonists for endocrine sensitive cancers (e.g., prostate, endometrial, hormone receptor-positive breast cancer).

In another embodiment of the methods described herein, a patient described herein has not received prior treatment with H₂ blockers or antacids within 14 days or five drug-elimination half-lives, whichever is longer prior to the start of treatment as described herein.

In another embodiment of the methods described herein, a patient described herein has not received prior treatment with mild or moderate CYP3A4 inhibitors or mild or moderate CYP3A4 inducers within 14 days or five drug-elimination half-lives, whichever is longer prior to the start of treatment as described herein.

### Kits

The combination therapies described herein can be provided as a kit comprising one or more of the agents described herein for administration. In one embodiment, the kit includes Compound 1, or a pharmaceutically acceptable salt thereof, for administration in combination with atezolizumab as described herein. In another embodiment, the kit includes Compound 1, or a pharmaceutically acceptable salt thereof, packaged together with atezolizumab, wherein the kit comprises separate formulated dosages of each agent.

Also provided herein is an article of manufacture or a kit comprising Compound 1, or a pharmaceutically acceptable salt thereof, and a PD-L1 binding antagonist (e.g., atezolizumab). In some instances, the article of manufacture further comprises package insert comprising instructions for using the PD-L 1 binding antagonist to treat lung cancer, head and neck cancer, or melanoma as described herein. In one such embodiment, the lung cancer in NSCLC. In one such embodiment, the head and neck cancer is HNSCC. In one such embodiment, the melanoma is BRAFT WT melanoma. In one embodiment, the article of manufacture further comprises package insert comprising instructions for using atezolizumab in combination with Compound 1, or a pharmaceutically acceptable salt thereof, to treat NSCLC, HNSCC, or BRAFT WT melanoma as described herein in a patient described herein.

In some instances, the PD-L1 binding antagonist (e.g., atezolizumab) and Compound 1, or a pharmaceutically acceptable salt thereof, are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the articles of manufacture or kits described herein may include instructions to administer Compound 1, or a pharmaceutically acceptable salt thereof, and/or the PD-L1 binding antagonist (e.g., atezolizumab) to a patient as described herein in accordance with any of the methods described herein.

### Embodiments

Provided below are exemplary embodiments of the invention.
Embodiment 1. A combination therapy comprising:
   (a) Compound 1

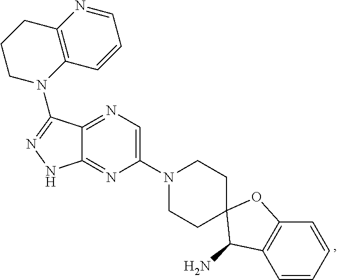 [SMILES: N[C@@H]1c2ccccc2OC12CCN(c1cnc3c(N4CCCc5ncccc54)n[nH]c3n1)CC2]

or a pharmaceutically acceptable salt thereof; and
   (b) a PD-L1 binding antagonist.
Embodiment 2. The combination therapy of embodiment 1, wherein the PD-L1 binding antagonist is an anti-PD-L1 antibody.
Embodiment 3. The combination therapy of embodiment 1 or 2, wherein the anti-PD-L1 antibody is atezolizumab.
Embodiment 4. The combination therapy of embodiment 3, wherein Compound 1, or a pharmaceutically acceptable salt thereof, is administered QD on days 1-21 of a first 21-day cycle and atezolizumab administered on day 1 of the first 21-day cycle.
Embodiment 5. The combination therapy of embodiment 3, wherein Compound 1, or a pharmaceutically acceptable salt thereof, is administered BID on days 1-21 of a first 21-day cycle and atezolizumab administered on day 1 of the first 21-day cycle.
Embodiment 6. The combination therapy of any one of embodiments Ito 5, wherein Compound 1, or a pharmaceutically acceptable salt thereof, is administered orally as a tablet or capsule.
Embodiment 7. The combination therapy of any one of embodiments 1 to 6, wherein Compound 1, or a pharmaceutically acceptable salt thereof, is administered at an amount of about 5 mg-100 mg.
Embodiment 8. The combination therapy of any one of embodiments 1 to 7, wherein Compound 1, or a pharmaceutically acceptable salt thereof, is administered at an amount of about 5 mg, 10 mg, 20 mg, 40 mg, 60 mg, 80 mg, or 100 mg.
Embodiment 9. The combination therapy of any one of embodiments 3 to 8, wherein atezolizumab is administered Q3W at an amount of about 800 mg to about 1400 mg.
Embodiment 10. The combination therapy of any one of embodiments 3 to 9, wherein atezolizumab is administered at an amount of about 1200 mg on day 1 of each cycle.
Embodiment 11. The combination therapy of any one of embodiments 5 to 10, wherein prior to the start of the first 21-day cycle, Compound 1, or a pharmaceutically acceptable salt thereof, is administered followed by atezolizumab administered at an amount of 840 mg.
Embodiment 12. The combination therapy of any one of embodiments 1 to 11, wherein the combination therapy is administered to a patient in need thereof, the patient having lung cancer, head and neck cancer, or melanoma.
Embodiment 13. The combination therapy of embodiment 12, wherein the patient is PD-L1 positive.
Embodiment 14. The combination therapy of embodiment 12 or 13, wherein the patient is PD-L1-high.
Embodiment 15. The combination therapy of any one of embodiments 12 to 14, wherein the patient is PD-L1-low.
Embodiment 16. The combination therapy of any one of embodiments 13 to 15, wherein the PD-L1 positivity is determined by a PD-L1 immunohistochemistry (IHC) assay.
Embodiment 17. The combination therapy of any one of embodiments 12 to 15, wherein the patient has non-small cell lung cancer (NSCLC), head and neck squamous cell carcinoma (HNSCC), or BRAF WT melanoma.
Embodiment 18. A method of treating lung cancer, head and neck cancer, or melanoma in a patient in need thereof, the method comprising administering during a treatment period an effective amount of a combination therapy comprising:
   (a) Compound 1

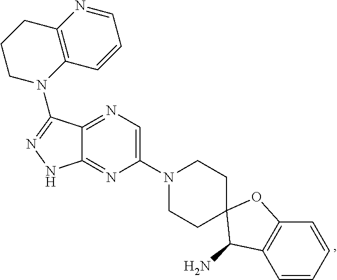 [SMILES: N[C@@H]1c2ccccc2OC12CCN(c1cnc3c(N4CCCc5ncccc54)n[nH]c3n1)CC2]

or a pharmaceutically acceptable salt thereof; and
   (b) a PD-L1 binding antagonist.
Embodiment 19. The method of embodiment 18, wherein the PD-L1 binding antagonist is an anti-PD-L1 antibody.
Embodiment 20. The method of embodiment 18, wherein the anti-PD-L1 antibody is atezolizumab.
Embodiment 21. The method of embodiment 20, wherein the treatment period comprises administering:
   (a) Compound 1, or a pharmaceutically acceptable salt thereof, QD on days 1-21 of a first 21-day cycle; and
   (b) atezolizumab on day 1 of the first 21-day cycle.
Embodiment 22. The method of embodiment 20, wherein the treatment period comprises administering:
   (a) Compound 1, or a pharmaceutically acceptable salt thereof, BID on days 1-21 of a first 21-day cycle; and
   (b) atezolizumab on day 1 of the first 21-day cycle.
Embodiment 23. The method of any one of embodiments 18 to 22, wherein Compound 1, or a pharmaceutically acceptable salt thereof, is administered orally as a tablet or capsule.
Embodiment 24. The method of any one of embodiments 18 to 23, wherein Compound 1, or a pharmaceutically acceptable salt thereof, is administered at an amount of about 5 mg-100 mg.
Embodiment 25. The method of any one of embodiments 18 to 24, wherein Compound 1, or a pharmaceutically acceptable salt thereof, is administered at an amount of about 5 mg, 10 mg, 20 mg, 40 mg, 60 mg, 80 mg, or 100 mg.
Embodiment 26. The method of any one of embodiments 20 to 25, wherein atezolizumab is administered Q3W at an amount of about 800 mg to about 1400 mg.
Embodiment 27. The method of any one of embodiments 20 to 26, wherein atezolizumab is administered at an amount of about 1200 mg on day 1 of each cycle.
Embodiment 28. The method of any one of embodiments 20 to 27, wherein the method further comprises a run-in period comprising administering prior to the start of the first cycle, Compound 1, or a pharmaceutically acceptable salt thereof, followed by atezolizumab administered at an amount of 840 mg.
Embodiment 29. The method of embodiment 28, wherein the run-in period comprises 1-14 days.
Embodiment 30. The method of embodiments 28 or 29, wherein the run-in period is 14 days and Compound 1, or a pharmaceutically acceptable salt thereof, and atezolizumab are administered on day 1 of the run-in period.
Embodiment 31. The method of embodiment 29 or 30, wherein Compound 1, or a pharmaceutically acceptable salt thereof, is administered QD starting on Day 8 of the run-in period.
Embodiment 32. The method of any one of embodiments 18 to 28, wherein the patient is PD-L1 positive.
Embodiment 33. The method of any one of embodiments 18-28 or 32, wherein the patient is PD-L1-high.
Embodiment 34. The method of any one of embodiments 18-28 or 32-33, wherein the patient is PD-L1-low.
Embodiment 35. The method of any one of embodiments 18-28 or 32-34, wherein the PD-L1 positivity is determined by a PD-L1 immunohistochemistrv (IHC) assay.
Embodiment 36. The method of any one of embodiments 18 to 35, wherein the lung cancer is non-small cell lung cancer (NSCLC).
Embodiment 37. The method of any one of embodiments 18 to 35, wherein the head and neck cancer is head and neck squamous cell carcinoma (HNSCC).
Embodiment 38. The method of any one of embodiments 18 to 35, wherein the melanoma is BRAF WT melanoma.
Embodiment 39. A method of treating lung cancer, head and neck cancer, or melanoma in a patient in need thereof, the method comprising administering to the patient a treatment regimen comprising an effective amount of Compound 1, or a pharmaceutically acceptable salt thereof, and a PD-L1 binding antagonist.
Embodiment 40. The method of embodiment 39, wherein the PD-L1 binding antagonist is an anti-PD-L1 antibody.
Embodiment 41. The method of embodiment 39 or 40, wherein the anti-PD-L1 antibody is atezolizumab.
Embodiment 42. The method of embodiment 41, wherein:
   (a) Compound 1, or a pharmaceutically acceptable salt thereof, is administered at an amount of about 5 mg-100 mg QD on days 1-21 of the first 21-day cycle; and
   (b) atezolizumab is administered at an amount of 1200 mg on day 1 of the first 21-day cycle.
Embodiment 43. The method of any one of embodiments 39 to 42, wherein Compound 1, or a pharmaceutically acceptable salt thereof, is administered at an amount of about 5 mg, 10 mg, 20 mg, 40 mg, 60 mg, 80 mg, or 100 mg.
Embodiment 44. The method of any one of embodiments 39 to 43, wherein the lung cancer is non-small cell lung cancer (NSCLC).
Embodiment 45. The method of any one of embodiments 39 to 43, wherein the head and neck cancer is head and neck squamous cell carcinoma (HNSCC).
Embodiment 46. The method of any one of embodiments 39 to 43, wherein the melanoma is BRAFT WT melanoma.
Embodiment 47. Use (U1) of a combination therapy comprising Compound 1, or a pharmaceutically acceptable salt thereof, and atezolizumab for the treatment of lung cancer, head and neck cancer, or melanoma as described herein.
Embodiment 48. The use of embodiment 47, further comprising a dosing regimen comprising: (a) administering Compound 1, or a pharmaceutically acceptable salt thereof, QD on days 1-21 of a first 21-day cycle; and (b) administering atezolizumab on day 1 of the first 21-day cycle.
Embodiment 49. The use of embodiment 47 or 48, further comprising (a) administering about 50 mg-500 mg of Compound 1, or a pharmaceutically acceptable salt thereof, QD on days 1-21 of the first 21-day cycle; and (b) administering about 1200 mg of atezolizumab on day 1 of the first 21-day cycle.
Embodiment 50. Use (U5) of a combination therapy comprising Compound 1, or a pharmaceutically acceptable salt thereof, and atezolizumab for the manufacture of a medicament for the treatment of lung cancer, head and neck cancer, or melanoma.
Embodiment 51. The use of embodiment 50, further comprising: (a) administering Compound 1, or a pharmaceutically acceptable salt thereof, QD on days 1-21 of a first 21-day cycle; and (b) administering atezolizumab Q3W on day 1 of the first 21-day cycle.
Embodiment 52. The use of embodiment 50 or 51, further comprising: (a) administering Compound 1, or a pharmaceutically acceptable salt thereof, QD on days 1-21 of a first 21-day cycle; and (b) administering atezolizumab on day 1 of the first 21-day cycle.
Embodiment 53. The use of any one of embodiments 50 to 52, further comprising: (a) administering about 50 mg-500 mg of Compound 1, or a pharmaceutically acceptable salt thereof, QD on days 1-21 of a first 21-day cycle; and (b) administering about 1200 mg atezolizumab on day 1 of the first 21-day cycle.
Embodiment 54. The use of any one of embodiments 50 to 53, wherein a dosing regimen includes two or more cycles.
Embodiment 55. The use of any one of embodiments 47 to 54, wherein Compound 1 is administered at an amount of about 5 mg-100 mg.
Embodiment 56. The use of any one of embodiments 47 to 55, wherein Compound 1 is administered at an amount of about 5 mg, 10 mg, 20 mg, 40 mg, 60 mg, 80 mg, or 100 mg.
Embodiment 57. The use of any one of embodiments 47 to 56, wherein the lung cancer is non-small cell lung cancer (NSCLC).
Embodiment 58. The use of any one of embodiments 47 to 56, wherein the head and neck cancer is head and neck squamous cell carcinoma (HNSCC).
Embodiment 59. The use of any one of embodiments 47 to 56, wherein the melanoma is BRAFT WT melanoma.
Embodiment 60. A combination therapy comprising Compound 1, or a pharmaceutically acceptable salt thereof, and atezolizumab for use in the treatment of lung cancer, head and neck cancer, or melanoma.
Embodiment 61. The combination therapy of embodiment 60, wherein: (a) Compound 1, or a pharmaceutically acceptable salt thereof, is administered QD on days 1-21 of a first 21-day cycle; and (b) atezolizumab is administered Q3W on day 1 of the first 21-day cycle.
Embodiment 62. The combination therapy of embodiment 60 or 61, wherein: (a) Compound 1, or a pharmaceutically acceptable salt thereof, is administered QD on days 1-21 of a first 21-day cycle; and (b) atezolizumab is administered on day 1 of the first 21-day cycle.
Embodiment 63. The combination therapy of any one of embodiments 60 to 62, wherein: (a) about 50 mg-500 mg of Compound 1, or a pharmaceutically acceptable salt thereof, is administered QD on days 1-21 of a first 21-day cycle; and (b) about 1200 mg atezolizumab is administered on day 1 of the first 21-day cycle.
Embodiment 64. The combination therapy of any one of embodiments 60 to 63, wherein a dosing regimen includes two or more cycles.
Embodiment 65. The combination therapy of any one of embodiments 60 to 64, wherein Compound 1 is administered at an amount of about 5 mg-100 mg.
Embodiment 66. The combination therapy of any one of embodiments 60 to 65, wherein Compound 1 is administered at an amount of about 5 mg, 10 mg, 20 mg, 40 mg, 60 mg, 80 mg, or 100 mg.
Embodiment 67. The combination therapy of any one of embodiments 60 to 66, wherein the lung cancer is non-small cell lung cancer (NSCLC).
Embodiment 68. The combination therapy of any one of embodiments 60 to 66, wherein the head and neck cancer is head and neck squamous cell carcinoma (HNSCC).
Embodiment 69. The combination therapy of any one of embodiments 60 to 66, wherein the melanoma is BRAFT WT melanoma.

The following examples are presented by way of illustration, not limitation.

### EXAMPLES

### Example 1: In vitro and In vivo characterization

Agents. Compound 1 was formulated in 50 mM Sodium Citrate buffer, pH3.0 at a concentration of 60 mg/kg. Anti-PD-L1 mAbs (Mu IgG2a anti-PD-L1, hereafter referred to as anti-PD-L1) and anti-gp120 isotype control (Mu IgG1 anti-gp120; hereafter referred to as Isotype Control) were formulated as a solution in buffer of 20 mM Histidine Acetate, 240 mM Sucrose, 0.02% Tween 20™, pH 5.5. An oral-dosed vehicle control was 50 mM Sodium Citrate buffer, pH3.0.

Cell Lines. The EMT6 mammary carcinoma cell line was cultured in vitro, harvested in log-phase growth, and resuspended in Hank's Balanced Salt Solution (HBSS) containing Matrigel™ (BD Biosciences; San Jose, CA) at a 1:1 ratio by volume for in vivo inoculation. Animals were each inoculated with 0.1×10⁶ cells in the mammary gland (5, left) in a volume of 100 mL.

Mice tumor models. 10.6 weeks old Balb/c mice bearing tumors in the range of 141-196 mm³ were evenly distributed into 4 study groups of 10 mice each. The mean tumor volume across all groups was 165 mm³ at the start of dosing. The mean starting weight was 19.94 g. All concentrations were calculated based on a mean body weight of 20 g and adjusted for 10% body weight loss or gain. Mice in the various groups received the treatment regimens described in Table 1. Mice were dosed for a total of 28 days or until euthanized at first sign of morbidity or if body weight loss was >20% of their weight at the start of administration.

The study extended beyond 28 days and following the cessation of treatment to check for the durability of response and ended at day 102 wherein remaining mice were all euthanized.

TABLE 1
Treatment regimens

| | | Dose | Dose | | | Dosing |
|---|---|---|---|---|---|---|
| | | Level | Volume | | | window |
| Grp # | Treatment | (mg/kg) | (ul) | Route | Schedule | (days) |
| 1 | Vehicle; Isotype gp120 mAb | 0, 10/5 | 200/100 | PO, | QD, BIW | 0-21 |
| | | | | IV/IP | | |
| 2 | Compound 1 | 60 | 100 | PO | QD, BIW | 0-27 |
| 3 | Anti-PD-L1 (6E11.1.9 | 10/5 | 100 | IV/IP | BIW | 0-24 |
| | LALAPG) | | | | | |
| 4 | Compound 1 + Anti-PD-L1 | 60, 10/5 | 200/100 | PO, | QD, BIW | 0-27 |
| | | | | IV/IP | | |

Tumor and Body Weight Measurements. Tumor volumes were measured in two perpendicular dimensions (length and width) using Ultra Cal-IV calipers (model 54-10-111; Fred V. Fowler Co.; Newton, MA) and tumor volume was calculated according to the following formula:

$$Tumor size ( {mm}^{3} ) = ( longer measurement \times shorter {measurement}^{2} ) \times 0.5$$

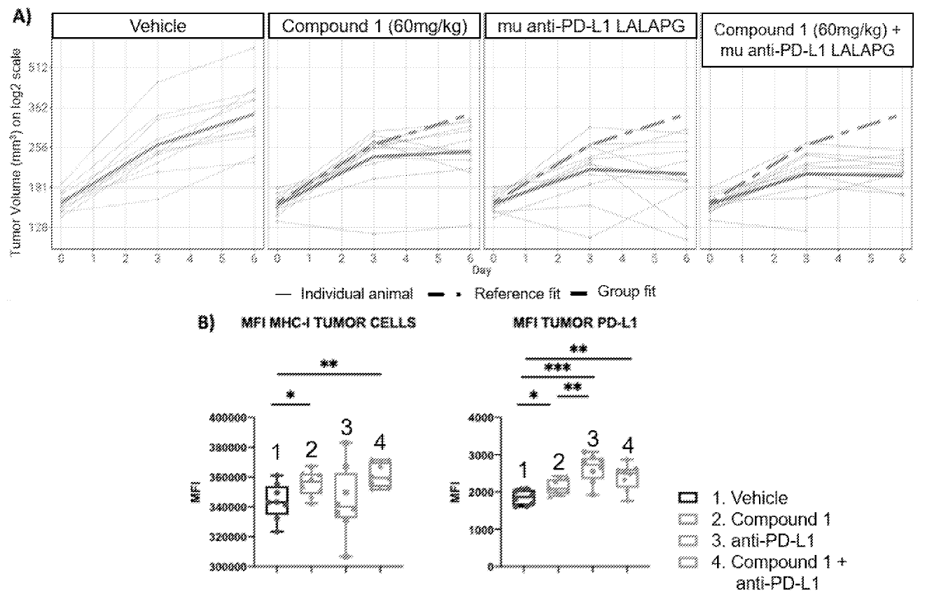

Tumors that regressed by >50% relative to their starting volume during the study were categorized as partial responders (PRs) while tumors with a final tumor volume corresponding to full regression were categorized as complete responders (CRs). Animal body weights were measured using an Adventura Pro AV812 scale (Ohaus Corporation; Pine Brook, NJ) and percent change in body weight for each group was calculated and plotted (see FIG. 4). Percent weight change was calculated using the following formula:

Body weight change (%)=[(current body weight/initial body weight)-1)×100]

Growth analysis and group comparisons. As tumors generally exhibit exponential growth, tumor volumes were subjected to natural log transformation before analysis. Analyses and comparisons of tumor growth were performed using a package of customized functions in R (Version 3.6.2); R Foundation for Statistical Computing; Vienna, Austria, which integrates software from open source packages (e.g., lme4, mgcv, gamm4, multcomp, settings, and plyr) and several packages from tidyverse (e.g., magrittr, dplyr, tidyr, and ggplot2) (Forrest et al.; Cancer Res 2020; 80(22):5089-97). All raw tumor volume measurements from 0 (minimum) to 8 mm³ (maximum value) were judged to reflect complete tumor absence and were converted to 8 mm³ prior to natural log transformation. A generalized additive mixed model (GAMM) was then applied to describe the changes in transformed tumor volumes over time using regression splines with automatically generated spline bases. This approach addresses both repeated measurements from the same study subjects and moderate dropouts before the end of the study.

Percent tumor growth inhibition (TGI) represents the percent difference between the area under the curve (AUC) for the treatment and reference group fits after back-transforming tumor volumes to the original scale, correcting for starting tumor burden, and averaging over a common time period. Positive values indicate anti-tumor effects, with 100% denoting stasis and values >100% denoting regression (negative values indicate a pro-tumor effect). The 95% confidence intervals are based on the fitted model and variability measures of the data, using parametric bootstrapping. Growth contrast represents the difference in area under the curve (AUC)-based growth rates (endpoint Gain Integrated in Time (eGalT)) between the treatment and reference group fits. To calculate AUC-based growth rates, group AUC values were corrected for starting tumor burden and subjected to slope equivalence normalization. When tumors show log-linear growth (i.e., the fit is a line on the natural log scale), slope equivalence normalization of the AUC results in the calculation of the slope of the fit. In cases wherein tumors demonstrate non-log-linear growth (i.e., the fit is curved on the natural log scale), slope equivalence normalization results in the calculation of the constant log-linear growth rate required to yield the observed baseline-corrected AUC for the fit. Mathematically, this normalization is attained by dividing each estimated baseline-corrected AUC value by half of the square of the common study period, resulting in units of natural log units per day. The vehicle control corresponds to a growth contrast (GR) value of 0. The more negative is the GR value, the greater is the anti-tumor effect. The 95% confidence intervals are based on the fitted model and variability measures of the data.

The growth rate of each individual tumor from the selected First Day AUC to the time of its final measurement is calculated from the AUC of the tumor volume profile in this time range after subjecting tumor volumes to natural log transformation first. To obtain the growth rate from the AUC, the AUC is corrected for starting tumor burden relevant to this period and then subjected to slope equivalence normalization. The baseline-corrected AUC value is divided by half of the square of the specified time period in this normalization step. The default units for this metric are natural log units per day as exponential growth on the original scale is linear on the natural log scale. Positive values denote growth, negative values denote regression, and 0 is indicative of stasis. The more positive a value, the faster a tumor is growing. The more negative a value, the faster a tumor is regressing. Individual AUC-based growth rates are not calculated for the purpose of group comparisons.

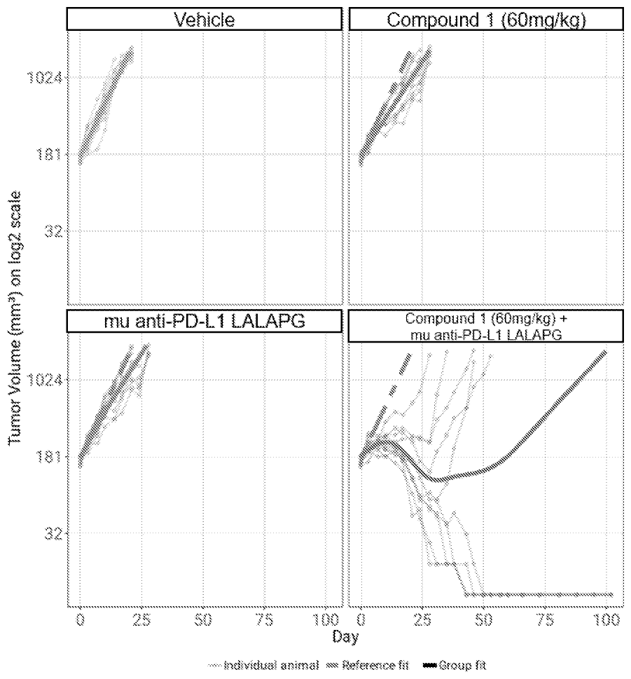

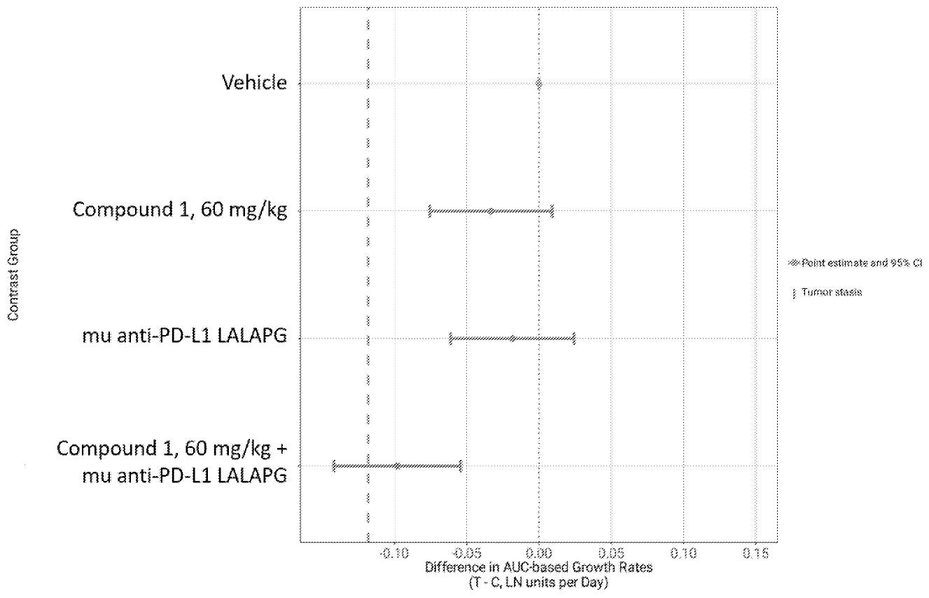

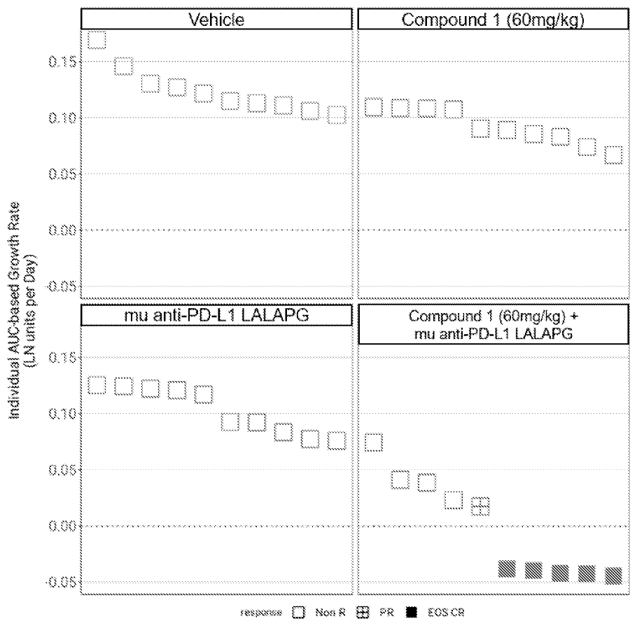

Anti-tumor efficacy was assessed in Balb/c mice bearing EMT6 mammary carcinoma following treatment with single agent of Compound 1 (60 mg/kg, PO, QD), anti-PD-L1 (10 mg/kg, IV, first dose, then 5 mg/kg, IP, twice weekly, or combination with of Compound 1 and anti-PD-L1. The single agent treatments resulted in no efficacy, with Compound 1 resulting in 47% TGI and −0.033 GR, and anti-PD-L1 resulting in 30% TGI and −0.022 GR, relative to vehicle controls. In contrast, the combination of Compound 1 with anti-PD-L1 exhibited an enhanced anti-tumor response close to tumor stasis following 20 days of dosing and resulting in 92% TGI and −0.098 GR, relative to vehicle controls. In addition, sustained anti-tumor efficacy was observed following the cessation of 28-day-dosing treatment with 1/10 PR and 5/10 CRs (see FIG. 1, FIG. 2, FIG. 3). All treatments were well tolerated, as determined by the percent change in body weights.

TABLE 2
Summary of the Effect of Compound 1 and Anti-PD-L1 Combination
Therapy on the EMT6 Mammary Carcinoma in BALB/c Mice

| | | Growth Contrast, LN | | Legacy % TGI | | | |
|---|---|---|---|---|---|---|---|
| Number | Group Name | (Lower, Upper) | | (Lower, Upper) | | PR | EOS CR |
| 1 | Vehicle | 0.000 | (0.000, 0.000) | 0 | (0, 0) | 0 (0.0%) | 0 (0.0%) |
| 2 | Compound 1 | −0.033 | (−0.075, 0.009) | 47 | (14, 67) | 0 (0.0%) | 0 (0.0%) |
| 3 | Anti-PD-L1 | −0.018 | (−0.061, 0.024) | 30 | (−8, 55) | 0 (0.0%) | 0 (0.0%) |
| 4 | Compound 1 + | −0.098 | (−0.142, −0.054) | 92 | (81, 103) | 1 (10.0%) | 5 (50.0%) |
| | anti-PD-L1 | | | | | | |

CI = confidence interval;
CR = complete responder;
PR = partial responder;
EOS = end of study;
TGI = tumor growth inhibition;
% TGI = percent of tumor growth inhibition based on AUC (see Data Analysis section for equation).
Vehicle control is 50 mM Sodium Citrate buffer, PH3.0

Tumor Immuno-profiling. Anti-tumor immunity was evaluated in EMT6 mammary tumors by flow cytometric immuno-profiling of tumor cells and tumor-infiltrating immune cells the tumor after 7-days of treatment with either vehicle and isotype control antibodies, single agent Compound 1 (60 mg/kg, PO, QD), anti-PD-L1 antibodies (10 mg/kg, IV, first dose, then 5 mg/kg, IP, twice per week), or the combination with of Compound 1 and anti-PD-L1.

Tumor cells (CD45 negative cells) assessed for expression of MHC-I and PD-L1 revealed that in the treated groups with Compound 1 alone and in combination with anti-PD-L1 there was a significant increase in mean fluorescing intensity (MFI) of both MHC-I (p=0.0354 and p=0.0044, respectively) and PD-L1 (p=0.0127 and p=0.0018, respectively) expression relative to those in the vehicle group (See FIG. 4).

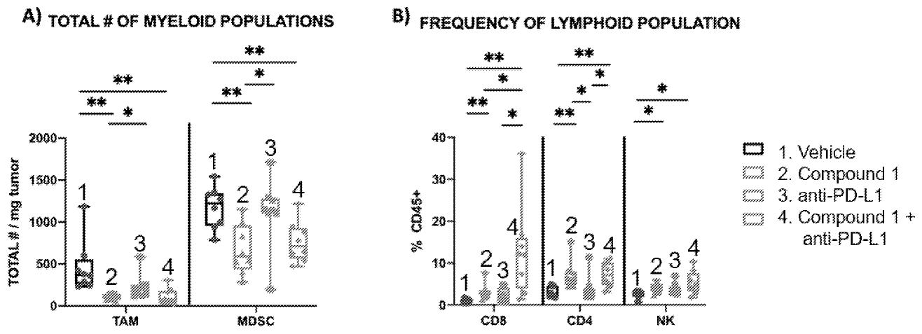

In tumors from mice treated with either Compound 1 alone or in combination with anti-PD-L1 antibodies there was a reduction in the frequency of MDSCs (p=0.0058 and p=0.0083, respectively) and TAMs (p=0.0072 and p=0.0098, respectively) relative to the vehicle group (FIG. 5A). Further, there was an increase in the frequency of the M1 population of macrophages (p=0.0033 and p=0.0162, respectively) with a compensatory decrease in M2 population of macrophages (p=0.0036 and p=0.0007, respectively) in the tumor microenvironment of mice treated with Compound 1 alone or in combination with anti-PD-L1 relative to the vehicle group, with an increase in the M1/M2 ratio in the combination (p=0.0033). Myeloid-derived dendritic cells (DCs) showed no changes in any treatment group. These data demonstrate that SHP2 inhibition by Compound 1 reduces tumor MDSCs and skews the phenotype of TAMs to become more pro-inflammatory.

Tumor-infiltrating lymphocytes (TILs) were assessed, including CD4+ and CD8+ T cells as well as NK cell populations, to determine the effect of SHP2 inhibition on T and NK cell activity. In the Compound 1 and anti-PD-L1 combination group there was an increased in mice treated with Compound 1 alone or in combination with anti-PDL1 antibodies compared to the vehicle group (CD8, p=0.0058 and p=0.008; CD4, p=0.0067 and p=0.002; NK, p=0.013 and p=0.024, respectively) (FIG. 5B).

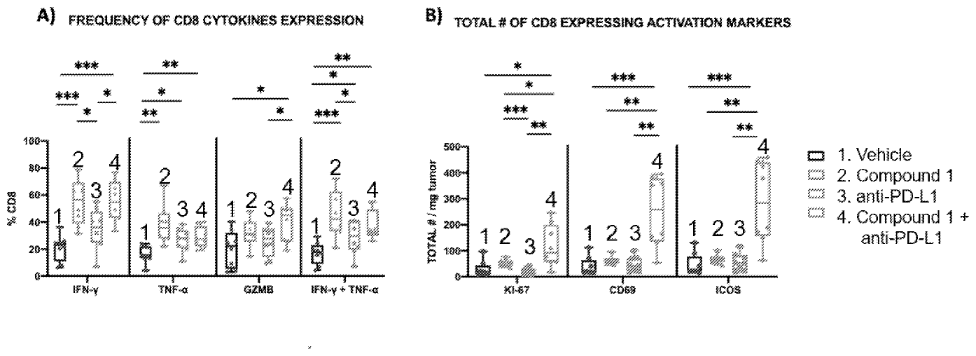

The activation status of the TILs was assessed by assessing the expression of cytokines and effector molecules, including interferon gamma (IFNg), tumor necrosis factor alpha (TNFa) and granzyme B (GZMB) from tumor-derived T cells under restimulation conditions using PMA/ionomycin (FIG. 6A). There was an increase in the expression of both IFNg and TNFa on tumor-derived CD8+ T cells from mice treated with Compound 1 alone or in combination with anti-PD-L1 antibodies relative to vehicle group (IFNg, p=0.0002 and p=0.0001; TNFa, p=0.0016 and p=0.0035, respectively) (FIG. 6A), consistent with these T cells being more active. There was also an increase of the frequency of CD8+ T cells expressing GZMB in tumors from mice treated with Compound 1 and anti-PD-L1 relative to the vehicle group (p=0.0137). (FIG. 6A). The polyfunctionality of tumor-infiltrating CD8⁺ T cells (expressing IFNg and TNFa) was increased when mice were treated with Compound 1 alone or in combination with anti-PD-L1 antibodies relative to the vehicle group (p=0.0008 and p=0.0011, respectively). Markers of T cell activation and exhaustion were also assessed to determine if SHP2 inhibition by Compound 1 alone or in combination with anti-PD-L1 antibodies altered the phenotype of CD8+ T cells in the tumor. Tumors from mice treated with Compound 1 and anti-PDL1 antibodies showed an increase in the total number of CD8⁺ T cells expressing the activation markers Ki-67 (p=0.0127), CD69 (p=0.0009) and ICOS (p=0.001) (FIG. 6B).

These data demonstrate that SHP2 inhibition by Compound 1 alone or in combination with anti-PD-L1 results in significant increased NK cell and CD4+ and CD8+ T cell activation in EMT6 tumors.

These studies collectively demonstrate that SHP2 inhibition by Compound 1 at 60 mg/kg, QD in combination with anti-PD-L1 antibodies demonstrates significant anti-tumor activity in the EMT6 syngeneic tumor model. In addition to the increased anti-tumor activity observed during the dosing interval of 28 days, there was also an increase in the number of durable responders with an 6/10 (60%) ORR and 5/10 (50%) CRs at the end of study (day 102). These data demonstrated that combining the SHP2 inhibitor Compound 1 with anti-PD-L 1 antibodies was a well-tolerated and active combination regimen in the EMT6 syngeneic tumor model.

These data demonstrate that combining the SHP2 inhibitor Compound 1 with anti-PD-L1 antibodies promotes an anti-tumor immune response through pharmacodynamic changes observed in tumor cells as well as the tumor microenvironment that collectively promote deep and durable tumor inhibition in a significant number of animals relative to single agents alone.

### Example 2

Compound 1 is a potent allosteric inhibitor of recombinant human SHP2, with inhibitory concentration of 50% (IC50) of 0.7 nM. It has been characterized in numerous nonclinical cancer models. Compound 1 has also been demonstrated to be a potent inhibitor of SHP2 in cell-based assays. SHP2 inhibition in cells was determined by measuring the level of ERKT/2 phosphorylation at Thr202/Tyr204 with an IC50 value of 1.3 nM in EGFR-amplified KYSE-520 cells. A comprehensive evaluation of selectivity against a panel of kinases, receptors, ion channels, and transporters demonstrated that Compound 1 selectively inhibits SHP2.

The anti-proliferative effect of Compound 1 has been assessed in multiple cancer cell lines using 2D and anchorage-independent 3D formats. Among panels of NSCLC, esophageal squamous cell carcinoma, colorectal carcinoma, pancreatic ductal adenocarcinoma, cholangiocarcinoma, and other tumor cell lines tested in 2D or 3D assays, Compound 1 inhibited the proliferation of numerous cell lines at IC50 values below 1 nM.

Anti-proliferative effects were seen in cell lines harboring various aberrations in the RAS/MAPK signaling pathway. These in vitro results suggest Compound 1 has potential for broad anti-tumor effects across a variety of tumor types.

Compound 1 has also been tested for in vivo anti-tumor activity in multiple xenograft models. Oral administration of single-agent Compound 1 demonstrated significant anti-tumor efficacy in human tumor xenograft models of, for example, KRAS-mutant NSCLC (NCI-H358) and EGFR-amplified esophageal squamous cell carcinoma (KYSE-520). Dose-dependent anti-tumor activity was observed at dose levels that were well tolerated.

Pharmacokinetic, pharmacodynamic, and efficacy comparisons showed that the extent and duration of SHP2 inhibition was directly correlated to the level of anti-tumor activity.

Collectively, these in vitro and in vivo studies support the clinical evaluation of Compound 1 as an anti-tumor agent.

Atezolizumab is a humanized IgG1 monoclonal antibody that targets PD-L1 and inhibits the interaction between PD-L1 and its receptors, PD-1 and B7-1 (also known as CD80), both of which function as inhibitory receptors expressed on T cells. Therapeutic blockade of PD-L1 binding by atezolizumab has been shown to enhance the magnitude and quality of tumor-specific T-cell responses, resulting in improved anti-tumor activity.

Atezolizumab has minimal binding to Fc receptors, thus eliminating detectable Fc effector function and associated antibody-mediated clearance of activated effector T cells.

Atezolizumab shows anti-tumor activity in both nonclinical models and cancer patients and is being investigated as a potential therapy in a wide variety of malignancies.

Atezolizumab is being studied as a single agent in the advanced cancer and adjuvant therapy settings, as well as in combination with chemotherapy, targeted therapy, and cancer immunotherapy. Atezolizumab is approved (as a single agent and/or in combination with other anti-cancer therapies) for the treatment of locally advanced or metastatic urothelial carcinoma, NSCLC, small-cell lung cancer, triple-negative breast cancer, melanoma, and hepatocellular carcinoma.

Rationale for Combination Therapy with Atezolizumab. Encouraging clinical data emerging in the field of tumor immunotherapy have demonstrated that therapies focused on enhancing T-cell responses against cancer can result in a significant survival benefit in patients with advanced malignancies. The PD-L1 pathway serves as an immune checkpoint to temporarily dampen immune responses in states of chronic antigen stimulation, such as chronic infection or cancer. Interruption of the PD-L1 pathway represents one path for restoring tumor-specific T-cell immunity.

In nonclinical pharmacodynamic (PD) and efficacy studies, the SHP2 inhibitor Compound 1 (at 60 mg QD) in combination with checkpoint inhibition (anti-PD-L1) produced an increase in the inflammatory state of the tumor microenvironment in the EMT6 breast cancer syngeneic model, enabling effective engagement of tumors by the immune system that results in stronger anti-tumor response. (See Example 1.) In tumor cells, Compound 1 plus anti-PD-L1 increased tumor MHC I and PD-L1 expression. Compound 1 alone or in combination with anti-PD-L1 led to a reduction in MDSCs and TAMs. In addition, the remaining TAMs were skewed towards the pro-inflammatory M1 phenotype. Compound 1 alone and in combination with anti-PD-L1 also drove an increase in CD4⁺ and CD8⁺ T cells as well as NK cells present in tumors. The combination of Compound 1 and anti-PD-L 1 drove an increase in the activation state of CD8⁺ T cells in the tumor, including an increase in proliferative markers Ki-67 and CD69 and T-cell maturation marker ICOS1. Compound 1 and anti-PD-L1 show strong combination efficacy in the EMT6 tumor model, resulting in deeper and more durable responses relative to either single agent.

High PD-L1 expression can be predictive of response to immune checkpoint inhibitor (CPI) therapy. With its ability to facilitate increased PD-L1 expression, antigen presentation on tumor cells, and enhanced infiltration and activation of CD8⁺ T cells, SHP2 inhibition can synergize with PD-L1 blockade in tumors. Atezolizumab has demonstrated activity in patients with advanced malignancies. Objective responses have been observed across a broad range of malignancies, including for example, lung cancer (e.g., NSCLC), urothelial carcinoma, RCC, melanoma (e.g., BRAFT WT melanoma), colorectal cancer, head and neck cancer (e.g., HNSCC), gastric cancer, breast cancer, and sarcoma.

Patients with disease-specific malignancies that are responsive to anti-PD-1/PD-L1 activity will likely benefit from the combination therapies described herein. PD-L1-positive NSCLC and PD-L1-positive metastatic head and neck squamous cell carcinoma (HNSCC) in the first-line treatment setting are responsive to anti-PD-1/PD-L1 agents and single-agent anti-PD-1/PD-L1 therapy is a recognized standard-of-care regimen shown to provide benefit. Although multiple treatment types are available for these diseases (e.g., (chemotherapy, immunotherapy), these patients have incurable disease representing a high unmet need.

Patients with BRAF^{V600} wild type (BRAF WT) melanoma in the second-line or later metastatic treatment setting will also be treated. These patients have incurable disease, and there are no approved treatments following the use of anti-PD-L1 therapy, with or without anti-CTLA4 treatment. Typically, these patients may be treated with additional CPI treatment.

Studies have shown a range of response rates (4% to 36%), indicating re-treatment may provide some limited benefit. The addition of Compound 1 to atezolizumab may provide additional anti-tumor activity.

Atezolizumab has been generally well tolerated. Adverse events with potentially immune-mediated causes consistent with an immunotherapeutic agent, including rash, influenza-like illness endocrinopathies, hepatitis or transaminitis, pneumonitis colitis, and myasthenia gravis, have been observed.

Compound 1 and atezolizumab will be administered until unacceptable toxicity or loss of clinical benefit as determined by the investigator after an integrated assessment of radiographic data and clinical status (e.g., symptomatic deterioration such as pain secondary to disease). Treatment with Compound 1 and atezolizumab may continue beyond apparent radiographic disease progression.

In one instance, the treatment with the Compound 1 and atezolizumab combination described herein will consist of a 14-day run-in period followed by a treatment period as described herein. During the run-in period, patients will receive treatment on Days 1 and 8, as outlined below:
Day 1: Compound 1 tablet 60 mg PO QD followed by atezolizumab 840 mg IV
Day 8: Compound 1 capsule 60 mg PO QD

Upon completion of the 14-day run-in period, patients will enter the treatment period. Starting on Day 1 of Cycle 1, patients will receive treatment in 21-day cycles, as outlined below:
Compound 1 capsule (at assigned dose e.g., 20 mg, 40 mg, 60 mg) PO QD on Days 1-21 of each cycle
Atezolizumab 1200 mg IV on Day 1 of each cycle

Activity of Compound 1 in combination with atezolizumab will be assessed according to RECIST v1.1. for ORR, DOR, PFS, PFS rate, and OS rate).

Inclusion Criteria: Patients must meet the following criteria for treatment as described herein:
Age ≥18 years at time of signing Informed Consent Form
Measurable disease per RECIST v1.1
ECOG Performance Status of 0 or 1
Life expectancy >12 weeks
Adequate hematologic and end-organ function obtained within 14 days prior to initiation of treatment:
   ANC ≥1.5×10⁹/L (1500/mL)
   WBC ≥2,500/mL
   Lymphocyte count ≥0.5×10⁹/L (500/mL)
   Platelet count ≥100×10⁹/L (100,000/mL)
   Hemoglobin ≥90 g/L (9 g/dL)
   Serum albumin ≥25 g/L (2.5 g/dL)
   AST, ALT, and ALP ≤2.5×ULN, with the following exceptions:
      Patients with documented liver metastases: AST and ALT ≤5×ULN
      Patients with documented liver or bone metastases: ALP 5 5×ULN
   Total bilirubin <1.5×ULN with the following exception:
      Patients with known Gilbert disease: total bilirubin ≤3×ULN
   Measured or calculated creatinine clearance ≥50 mL/min (calculated through use of the Cockcroft-Gault formula):

$$\frac{( 140 - age ) \times ( weight in kgs ) \times ( 0.85 if female )}{72 \times ( serum creatinine in mg / dL )}$$

Negative HIV test at screening
Negative hepatitis B surface antigen (HBsAg) test at screening
Positive hepatitis B surface antibody (HBsAb) test at screening, or negative HBsAb at screening accompanied by either of the following:
   Negative total hepatitis B core antibody (HBcAb)
   Positive total HBcAb test followed by quantitative hepatitis B virus (HBV) DNA <500 IU/mL
   The HBV DNA test must be performed for patients who have a negative HBsAg test, a negative HBsAb test, and a positive total HBcAb test.
Negative hepatitis C virus (HCV) antibody test at screening, or positive HCV antibody test followed by a negative HCV RNA test at screening
   The HCV RNA test must be performed for patients who have a positive HCV antibody test.
Histologically confirmed locally advanced or metastatic solid tumor that has progressed after at least one available standard therapy or for which approved standard therapy has proven to be ineffective or intolerable, standard therapy is considered inappropriate, or an investigational agent is a recognized standard of care

Additional Inclusion Criteria for NSCLC
Histologically confirmed locally advanced or metastatic NSCLC
No prior systemic therapy for locally advanced or metastatic NSCLC (adjuvant therapy is permitted)
Availability of a representative tumor specimen that is suitable for determination of EGFR and ALK alterations and PD-L1 status as assessed by a central or local laboratory testing
Absence of EGFR and ALK alterations or other known oncogenic driver mutations for which patients will have access to targeted therapeutic agents
PD-L1 positive

Additional Inclusion Criteria for HNSCC
Histologically confirmed, recurrent or metastatic HNSCC involving the oropharynx, oral cavity, larynx, or hypopharynx that is considered not amenable to curative therapy
Known results from local human papillomavirus (HPV) status test for oropharyngeal carcinoma
No prior systemic therapy for recurrent or metastatic HNSCC
Availability of a representative tumor specimen that is suitable for determination of PD-L1 status as assessed by a central or local laboratory IHC-based testing
PD-L1 positive

Additional Inclusion Criteria for Melanoma
Histologically confirmed locally advanced or metastatic (recurrent or de novo Stage IV) or unresectable locally advanced (Stage III) cutaneous BRAF WT melanoma (as documented by a local validated test at a Clinical Laboratory Improvement Amendments (CLIA) or equivalently certified laboratory), as defined by the American Joint Committee on Cancer 8th edition, that has progressed on or after treatment that included anti-PD-1 or anti-PD-L1 therapy
Patients must have progressive disease at study entry.

Exclusion Criteria: Patients who meet any of the following criteria will be excluded:
Symptomatic, untreated, or actively progressing central nervous system (CNS) metastases
Asymptomatic patients with treated CNS lesions are eligible, provided that all of the following criteria are met:
   Measurable disease, per RECIST v1.1, must be present outside the CNS.
   The patient has no history of intracranial hemorrhage or spinal cord hemorrhage.
   The patient has not undergone stereotactic radiotherapy within 7 days prior to treatment, whole-brain radiotherapy within 14 days prior to treatment, or neurosurgical resection within 28 days prior to treatment
   No ongoing requirement for corticosteroids as therapy for CNS disease.
   If the patient is receiving anticonvulsant therapy, the dose is considered stable.
   Metastases are limited to the cerebellum or the supratentorial region (i.e., no metastases to the midbrain, pons, medulla, or spinal cord).
   There is no evidence of interim progression between completion of CNS-directed therapy and initiation of study treatment.
Leptomeningeal disease or carcinomatous meningitis
History or evidence of ophthalmic disease including retinal vein occlusion (RVO), central serous retinopathy (CSR), predisposing factors to RVO or CSR, or retinopathy at the screening ophthalmic examination
Uncontrolled hypertension
Left ventricular ejection fraction <institutional lower limit of normal or <50%
History of severe allergic anaphylactic reactions to chimeric or humanized antibodies or fusion proteins
Known hypersensitivity to Chinese hamster ovary cell products or to any component of the atezolizumab formulation
Known allergy or hypersensitivity to any component of the Compound 1 formulation
Uncontrolled pleural effusion, pericardial effusion, or ascites requiring recurrent drainage procedures (once monthly or more frequently)
Clinically significant history of liver disease including viral or other hepatitis, current alcohol abuse, or cirrhosis
Uncontrolled or symptomatic hypercalcemia (ionized calcium >1.5 mmol/L, calcium >12 mg/dL, or corrected calcium >ULN)
Active or history of autoimmune disease or immune deficiency, including, but not limited to, myasthenia gravis, myositis, autoimmune hepatitis, systemic lupus erythematosus, rheumatoid arthritis, inflammatory bowel disease, anti-phospholipid antibody syndrome, Wegener granulomatosis, Sjogren syndrome, Guillain-Barre syndrome, or multiple sclerosis, with the following exceptions:
   Patients with a history of autoimmune-related hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met:
      Rash must cover <10% of body surface area
      Disease is well controlled at baseline and requires only low-potency topical corticosteroids
      There has been no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan
History of radiation pneumonitis in the radiation field (fibrosis) is permitted
Active tuberculosis
Major surgical procedure, other than for diagnosis, within 4 weeks prior to treatment
Prior allogeneic stem cell or solid organ transplantation
Current treatment with anti-viral therapy for HBV
Treatment with chemotherapy, immunotherapy, biologic therapy, or an investigational agent as anti-cancer therapy within 3 weeks or 5 drug-elimination half-lives prior to treatment, whichever is shorter; or endocrine therapy within 2 weeks prior to treatment, except for hormonal therapy with gonadotropin-releasing hormone agonists or antagonists for endocrine-sensitive cancers (e.g., prostate, endometrial, hormone receptor-positive breast cancer)
Treatment with strong CYP3A4 inhibitors or strong CYP3A4 inducers within 14 days or 5 drug-elimination half-lives, whichever is longer, prior to treatment
Treatment with therapeutic oral or IV antibiotics within 2 weeks prior to initiation of study drug
History of malignancy within 5 years prior to screening, with the exception of the cancer under investigation in this study and malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer
History or presence of an abnormal ECG
Treatment with a live, attenuated vaccine within 4 weeks prior to treatment
QT interval corrected through use of Fridericia's formula (QTcF) >440 ms demonstrated by at least two ECGs >30 minutes apart
History of ventricular dysrhythmias or risk factors for ventricular dysrhythmias such as structural heart disease (e.g., severe left ventricular systolic dysfunction, left ventricular hypertrophy), coronary heart disease (symptomatic or with ischemia demonstrated by diagnostic testing), clinically significant electrolyte abnormalities (e.g., hypokalemia, hypomagnesemia, hypocalcemia). or family history of sudden unexplained death or long QT syndrome
Current treatment with medications that are well known to prolong the QT interval
Treatment with H2 blockers or antacids within 14 days or 5 drug-elimination half-lives, whichever is longer, prior to initiation of study drug
Treatment with mild or moderate CYP3A4 inhibitors or mild or moderate CYP3A4 inducers (see details in Section 4.4.2.4) within 14 days or 5 drug-elimination half-lives,
whichever is longer, prior to treatment Treatment with any herbal therapies within 14 days or 5 drug-elimination half-lives, whichever is longer, prior to treatment

Patients with any history of immune deficiencies or autoimmune disease listed in the table below are excluded from participating in the study. Possible exceptions to this exclusion could be patients with a medical history of such entities as atopic disease or childhood arthralgias, wherein the clinical suspicion of autoimmune disease is low. Patients with a history of autoimmune-related hypothyroidism on a stable dose of thyroid replacement hormone may be eligible for this study. In addition, transient autoimmune manifestations of an acute infectious disease that resolved upon treatment of the infectious agent are not excluded (e.g., acute Lyme arthritis).

Autoimmune Diseases and Immune Deficiencies

Acute disseminated encephalomyelitis
Addison's disease
ANKA positive vasculitis
Ankylosing spondylitis
Antiphospholipid antibody syndrome
Aplastic anemia
Autoimmune hemolytic anemia
Autoimmune hepatitis
Autoimmune hypoparathyroidism
Autoimmune hypophysitis
Autoimmune myocarditis
Autoimmune oophoritis
Autoimmune orchitis
Autoimmune thrombocytopeni purpura
Behcet's disease
Bullous pemphigold
Celiac disease
Chronic fatigue syndrome
Chronic inflammatory demyelinating polyneuropathy
Chung-Strauss syndrome
Crohn's disease
Dermatomyositis
Diabetes mellitus type 1
Dysautonomia
Epidermolysis bullosa acquista
Gestational pemphigoid
Giant cell arteritis
Glomerulonephritis
Goodpasture's syndrome
Graves' disease
Guillain-Barré syndrome
Hashimoto's disease
IgA nephropathy
Inflammatory bowel disease
Interstitial cystitis
Kawasaki's disease
Lambert-Eaton myasthenia syndrome
Lupus erythematosus
Lyme disease - chronic
Meniere's syndrome
Mixed connective tissue disease
Mooren's ulcer
Morphea
Multiple sclerosis
Myasthenia gravis
Neuromyotonia
Opsoclonus myoclonus syndrome
Optic neuritis
Ord's thyroiditis
Pemphigus
Pernicious anemia
Polyarteritis nodusa
Polyarthritis
Polychondritis
Polyglandular autoimmune syndrome
Polymyositis
Primary biliary cirrhosis
Psoriasis
Reiter's syndrome
Pyoderma gangrenosum
Reactive arthritis
Rheumatoid arthritis
Sarcoidosis
Scleroderma
Sjogren's syndrome
Stiff-Person syndrome
Takayasu's arteritis
Ulcerative colitis
Vitiligo
Vogt-Kovanagi-Harada disease
Wegener's granulomatosis

Study Treatment Formulation, Packaging, and Handling. Compound 1 will be supplied as capsules (10 mg and 25 mg) and tablets (20 mg). Compound 1 will be administered orally in capsule or tablet form. During the treatment period, Compound 1 should be taken at approximately the same time each day on Days 1-21 of each 21-day cycle. The capsule or tablet should be swallowed whole and should not be chewed, crushed, or opened. A missed dose may be taken up to 4 hours following the planned dosing time. If a dose of Compound 1 is missed (i.e., not taken within 4 hours after the scheduled dosing time) or if vomiting occurs when the dose is taken, dosing should be resumed at the next scheduled dose.

Unless otherwise instructed, Compound 1 should be taken in the fasted state (1 hour before or 2 hours after a meal or snack) with approximately 240 mL of water. Patients will self-administer on an outpatient basis. Patients will receive Compound 1 until unacceptable toxicity or loss of clinical benefit as determined by the investigator after an integrated assessment of radiographic data and clinical status (see Section 3.1.3 for details).

Atezolizumab will be supplied as an IV formulation in 1200 mg/20 mL vials. Atezolizumab will be administered by IV infusion at a fixed dose of 1200 mg on Day 1 of each 21-day cycle, following administration of Compound 1. The start of the atezolizumab administration should be about 30 minutes after the oral administration of Compound 1. Administration of atezolizumab will be performed in a monitored setting wherein there is immediate access to trained personnel and adequate equipment and medicine to manage potentially serious reactions. Atezolizumab infusions will be administered per the instructions outlined in herein. No dose modification for atezolizumab is allowed.

Wherein a patients described herein participates in a 14-day run-in period as described herein, such patients will receive atezolizumab administered by IV infusion at a fixed dose of 840 mg on Day 1 of the run-in period. During the treatment periods of the dose-finding and expansion stages, all patients will receive atezolizumab administered by IV infusion at a fixed dose of 1200 mg on Day 1 of each 21-day cycle until unacceptable toxicity or loss of clinical benefit.

Atezolizumab will be administered following administration of Compound 1. Atezolizumab infusion should start 30 minutes after administration of Compound 1.

Administration of atezolizumab will be performed in a monitored setting wherein there is immediate access to trained personnel and adequate equipment and medicine to manage potentially serious reactions. Atezolizumab infusions will be administered per the instructions outlined in Table 3.

Administration of First and Subsequent Atezolizumab Infusions

| First Infusion | Subsequent Infusions |
|---|---|
| No premedication is permitted prior to the | If the patient experienced an infusion-related |
| atezolizumab infusion. | reaction with any previous infusion, |
| Vital signs (pulse rate, respiratory rate, blood | premedication with antihistamines, |
| pressure, and temperature) should be measured | antipyretics, and/or analgesics may be |
| within 60 minutes prior to the infusion. | administered for subsequent doses at the |
| Atezolizumab should be infused over 60 (±15) | discretion of the investigator. |
| minutes. | Vital signs should be measured within 60 |
| If clinically indicated, vital signs should be | minutes prior to the infusion. |
| measured every 15 (±5) minutes during the | Atezolizumab should be infused over 30 (±10) |
| infusion and at 30 (±10) minutes after the | minutes if the previous infusion was |
| infusion. | tolerated without an infusion-related reaction, |
| Patients should be informed about the possibility | or 60 (±15) minutes if the patient |
| of delayed post-infusion symptoms and instructed | experienced an infusion-related reaction with |
| to contact their study physician if they develop | the previous infusion. |
| such symptoms. | If the patient experienced an infusion-related |
| | reaction with the previous infusion or if |
| | clinically indicated, vital signs should be |
| | measured during the infusion and at 30 (±10) |
| | minutes after the infusion. |

Permitted Therapy. Patients are permitted to use the following therapies during the study:
Oral contraceptives
Hormone-replacement therapy
Prophylactic or therapeutic anticoagulation therapy (such as warfarin at a stable dose or low-molecular-weight heparin)
Vaccinations, however, live, attenuated vaccines are not permitted
Megestrol acetate
Mineralocorticoids (e.g., fludrocortisone)
Inhaled or low-dose corticosteroids
Low-dose corticosteroids
Palliative radiotherapy (e.g., treatment of known bony metastases or symptomatic relief of pain) as outlined below:
Premedication with antihistamines, antipyretics, and/or analgesics may be administered for the second and subsequent atezolizumab infusions only, at the discretion of the investigator.

Patients who experience infusion-associated symptoms may be treated symptomatically with acetaminophen, ibuprofen, diphenhydramine, and/or H₂-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and b₂-adrenergic agonists).

Cautionary Therapy. Systemic corticosteroids, immunosuppressive medications, and TNF-a inhibitors may attenuate potential beneficial immunologic effects of treatment with atezolizumab. Therefore, in situations in which systemic corticosteroids, immunosuppressive medications, or TNF-a inhibitors would be routinely administered, alternatives, including antihistamines, should be considered.

Prohibited Therapy. Use of the following concomitant therapies is prohibited as described below:
Investigational therapy (other than protocol-mandated study treatment) is prohibited within 3 weeks or 5 drug-elimination half-lives prior to treatment.
Concomitant therapy intended for the treatment of cancer (including, but not limited to, chemotherapy, hormonal therapy, immunotherapy, radiotherapy, and herbal therapy), whether health authority-approved or experimental, is prohibited prior to and during treatment.
Live, attenuated vaccines
Systemic immunostimulatory agents (including, but not limited to, interferons and IL-2)
Proton-pump inhibitors
Cannabidiol products
Strong CYP3A4 inhibitors or strong CYP3A4 inducers

For all patients in the run-in period, use of the following concomitant therapies is prohibited within 14 days or 5 drug-elimination half-lives, whichever is longer, prior to the run-in period:
H₂ blockers and antacids
Mild or moderate CYP3A4 inhibitors or mild or moderate CYP3A4 inducers
All herbal therapies

Risks Associated with Compound 1. Administration of Compound 1 has been associated with reversible and monitorable events including: Peripheral Edema, Myelosuppression (e.g., Thrombocytopenia, Anemia), Gastrointestinal Toxicities (e.g., Diarrhea, Nausea, and Vomiting), Elevation of Hepatic Transaminase, and Rash.

Risks associated with MAPK-Pathway Class Inhibitors. Administration of MAPK-pathway inhibitors has been associated with cardiomyopathy, retinopathy, Cardiomyopathy, CPK increase, thromboembolic events, Phototoxicity, and Reproductive Toxicity. Nonclinical safety studies with Compound 1 did not show evidence of cardiac, pulmonary, renal, thromboembolic, or ophthalmic toxicities.

Risks Associated with Atezolizumab. Atezolizumab has been associated with risks such as the following: infusion related reactions (IRRs) and immune-mediated hepatitis, pneumonitis, colitis, pancreatitis, diabetes mellitus, hypothyroidism, hyperthyroidism, adrenal insufficiency, hypophysitis, Guillain-Barre syndrome, myasthenic syndrome or myasthenia gravis, meningoencephalitis, myocarditis, nephritis, and myositis. Immune-mediated reactions may involve any organ system and may lead to hemophagocytic lymphohistiocytosis (HLH) and macrophage activation syndrome (MAS).

Adverse Events. An adverse event is defined herein to refer to any untoward medical occurrence in a clinical investigation subject administered an agent described herein in the combination therapies dh, regardless of causal attribution. The terms “severe” and “serious” are not synonymous. Severity refers to the intensity of an adverse event (e.g., rated as mild, moderate, or severe, or according to NCI CTCAE; see Section 5.3.3); the event itself may be of relatively minor medical significance (such as severe headache without any further findings).

Adverse events to be monitored include peripheral edema, diarrhea, dyspnea, hepatitis or elevation in ALT or AST, pneumonitis, any thromboembolic event, systemic lupus erythematosus, events suggestive of hypersensitivity (e.g., IRRs, CRS, HLH, and MAS), nephritis, ocular toxicities (e.g., uveitis, retinitis, optic neuritis), cardiac disorders (e.g., atrial fibrillation, myocarditis, pericarditis), vasculitis, autoimmune hemolytic anemia, and severe cutaneous reactions (e.g., Stevens-Johnson syndrome, dermatitis bullous, toxic epidermal necrolysis).

Throughout this specification and the claims, the words “comprise,” “comprises,” and “comprising” are used in a non-exclusive sense, except wherein the context requires otherwise. It is understood that embodiments described herein include “consisting of” and/or “consisting essentially of” embodiments.

Wherein a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included in the smaller ranges is also encompassed herein, subject to any specifically excluded limit in the stated range. Wherein the stated range includes one or both of the limits, ranges excluding either or both of those included limits are also included herein.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A combination therapy comprising:
(a) Compound 1 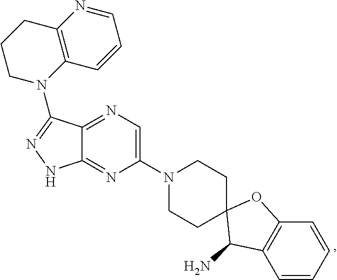 [SMILES: N[C@@H]1c2ccccc2OC12CCN(c1cnc3c(N4CCCc5ncccc54)n[nH]c3n1)CC2]
or a pharmaceutically acceptable salt thereof; and
(b) a PD-L1 binding antagonist.

2. The combination therapy of claim 1, wherein the PD-L1 binding antagonist is an anti-PD-L1 antibody.

3. The combination therapy of claim 1 or 2, wherein the anti-PD-L1 antibody is atezolizumab.

4. The combination therapy of claim 3, wherein Compound 1, or a pharmaceutically acceptable salt thereof, is administered QD on days 1-21 of a first 21-day cycle; and
atezolizumab administered on day 1 of the first 21-day cycle.

5. The combination therapy of claim 3, wherein Compound 1, or a pharmaceutically acceptable salt thereof, is administered BID on days 1-21 of a first 21-day cycle; and
atezolizumab administered on day 1 of the first 21-day cycle.

6. The combination therapy of any one of claims 1 to 5, wherein Compound 1, or a pharmaceutically acceptable salt thereof, is administered orally as a tablet or capsule.

7. The combination therapy of any one of claims 1 to 6, wherein Compound 1, or a pharmaceutically acceptable salt thereof, is administered at an amount of about 5 mg-100 mg.

8. The combination therapy of any one of claims 1 to 7, wherein Compound 1, or a pharmaceutically acceptable salt thereof, is administered at an amount of about 5 mg, 10 mg, 20 mg, 40 mg, 60 mg, 80 mg, or 100 mg.

9. The combination therapy of any one of claims 3 to 8, wherein atezolizumab is administered Q3W at an amount of about 800 mg to about 1400 mg.

10. The combination therapy of any one of claims 3 to 9, wherein atezolizumab is administered at an amount of about 1200 mg on day 1 of each cycle.

11. The combination therapy of any one of claims 5 to 10, wherein prior to the start of the first 21-day cycle, Compound 1, or a pharmaceutically acceptable salt thereof, is administered followed by atezolizumab administered at an amount of 840 mg.

12. The combination therapy of any one of claims 1 to 11, wherein the combination therapy is administered to a patient in need thereof, the patient having lung cancer, head and neck cancer, or melanoma.

13. The combination therapy of claim 12, wherein the patient is PD-L1 positive.

14. The combination therapy of claim 12 or 13, wherein the patient is PD-L1-high.

15. The combination therapy of any one of claims 12 to 14, wherein the patient is PD-L1-low.

16. The combination therapy of any one of claims 13 to 15, wherein the PD-L1 positivity is determined by a PD-L1 immunohistochemistry (IHC) assay.

17. The combination therapy of any one of claims 12 to 15, wherein the patient has non-small cell lung cancer (NSCLC), head and neck squamous cell carcinoma (HNSCC), or BRAF WT melanoma.

18. A method of treating lung cancer, head and neck cancer, or melanoma in a patient in need thereof, the method comprising administering during a treatment period an effective amount of a combination therapy comprising:
(a) Compound 1 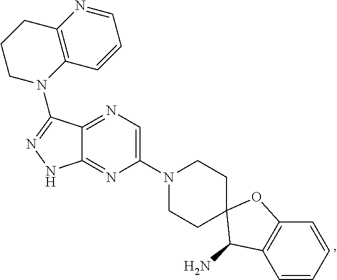 [SMILES: N[C@@H]1c2ccccc2OC12CCN(c1cnc3c(N4CCCc5ncccc54)n[nH]c3n1)CC2]
or a pharmaceutically acceptable salt thereof; and
(b) a PD-L1 binding antagonist.

19. The method of claim 18, wherein the PD-L1 binding antagonist is an anti-PD-L1 antibody.

20. The method of claim 18, wherein the anti-PD-L1 antibody is atezolizumab.

21. The method of claim 20, wherein the treatment period comprises administering:
(a) Compound 1, or a pharmaceutically acceptable salt thereof, QD on days 1-21 of a first 21-day cycle; and
(b) atezolizumab on day 1 of the first 21-day cycle.

22. The method of claim 20, wherein the treatment period comprises administering:
(a) Compound 1, or a pharmaceutically acceptable salt thereof, BID on days 1-21 of a first 21-day cycle; and
(b) atezolizumab on day 1 of the first 21-day cycle.

23. The method of any one of claims 18 to 22, wherein Compound 1, or a pharmaceutically acceptable salt thereof, is administered orally as a tablet or capsule.

24. The method of any one of claims 18 to 23, wherein Compound 1, or a pharmaceutically acceptable salt thereof, is administered at an amount of about 5 mg-100 mg.

25. The method of any one of claims 18 to 24, wherein Compound 1, or a pharmaceutically acceptable salt thereof, is administered at an amount of about 5 mg, 10 mg, 20 mg, 40 mg, 60 mg, 80 mg, or 100 mg.

26. The method of any one of claims 20 to 25, wherein atezolizumab is administered Q3W at an amount of about 800 mg to about 1400 mg.

27. The method of any one of claims 20 to 26, wherein atezolizumab is administered at an amount of about 1200 mg on day 1 of each cycle.

28. The method of any one of claims 20 to 27, wherein the method further comprises a run-in period comprising administering prior to the start of the first cycle, Compound 1, or a pharmaceutically acceptable salt thereof, followed by atezolizumab administered at an amount of 840 mg.

29. The method of claim 28, wherein the run-in period comprises 1-14 days.

30. The method of claims 28 or 29, wherein the run-in period is 14 days and Compound 1, or a pharmaceutically acceptable salt thereof, and atezolizumab are administered on day 1 of the run-in period.

31. The method of claims 28 or 29, wherein Compound 1, or a pharmaceutically acceptable salt thereof, is administered QD starting on day 8 of the run-in period.

32. The method of any one of claims 18 to 28, wherein the patient is PD-L1 positive.

33. The method of any one of claims 18-28 or 32, wherein the patient is PD-L1-high.

34. The method of any one of claims 18-28 or 32-33, wherein the patient is PD-L1-low.

35. The method of any one of claims 18-28 or 32-34, wherein the PD-L1 positivity is determined by a PD-L1 immunohistochemistry (IHC) assay.

36. The method of any one of claims 18 to 35, wherein the lung cancer is non-small cell lung cancer (NSCLC).

37. The method of any one of claims 18 to 35, wherein the head and neck cancer is head and neck squamous cell carcinoma (HNSCC).

38. The method of any one of claims 18 to 35, wherein the melanoma is BRAF WT melanoma.

39. A method of treating lung cancer, head and neck cancer, or melanoma in a patient in need thereof, the method comprising administering to the patient a treatment regimen comprising an effective amount of Compound 1, or a pharmaceutically acceptable salt thereof, and a PD-L1 binding antagonist.

40. The method of claim 39, wherein the PD-L1 binding antagonist is an anti-PD-L1 antibody.

41. The method of claim 39 or 40, wherein the anti-PD-L1 antibody is atezolizumab.

42. The method of claim 41, wherein:
(a) Compound 1, or a pharmaceutically acceptable salt thereof, is administered at an amount of about 5 mg-100 mg QD on days 1-21 of the first 21-day cycle; and
(b) atezolizumab is administered at an amount of 1200 mg on day 1 of the first 21-day cycle.

43. The method of any one of claims 39 to 42, wherein Compound 1, or a pharmaceutically acceptable salt thereof, is administered at an amount of about 5 mg, 10 mg, 20 mg, 40 mg, 60 mg, 80 mg, or 100 mg.

44. The method of any one of claims 39 to 43, wherein the lung cancer is non-small cell lung cancer (NSCLC).

45. The method of any one of claims 39 to 43, wherein the head and neck cancer is head and neck squamous cell carcinoma (HNSCC).

46. The method of any one of claims 39 to 43, wherein the melanoma is BRAFT WT melanoma.

47. Use of a combination therapy comprising Compound 1, or a pharmaceutically acceptable salt thereof, and atezolizumab for the treatment of lung cancer, head and neck cancer, or melanoma as described herein.

48. The use of claim 47, further comprising a dosing regimen comprising:
(a) administering Compound 1, or a pharmaceutically acceptable salt thereof, QD on days 1-21 of a first 21-day cycle; and
(b) administering atezolizumab on day 1 of the first 21-day cycle.

49. The use of claim 47 or 48, further comprising:
(a) administering about 50 mg-500 mg of Compound 1, or a pharmaceutically acceptable salt thereof, QD on days 1-21 of the first 21-day cycle; and
(b) administering about 1200 mg of atezolizumab on day 1 of the first 21-day cycle.

50. Use of a combination therapy comprising Compound 1, or a pharmaceutically acceptable salt thereof, and atezolizumab for the manufacture of a medicament for the treatment of lung cancer, head and neck cancer, or melanoma.

51. The use of claim 50, further comprising:
(a) administering Compound 1, or a pharmaceutically acceptable salt thereof, QD on days 1-21 of a first 21-day cycle; and
(b) administering atezolizumab Q3W on day 1 of the first 21-day cycle.

52. The use of claim 50 or 51, further comprising:
(a) administering Compound 1, or a pharmaceutically acceptable salt thereof, QD on days 1-21 of a first 21-day cycle; and
(b) administering atezolizumab on day 1 of the first 21-day cycle.

53. The use of any one of claims 50 to 52, further comprising:
(a) administering about 50 mg-500 mg of Compound 1, or a pharmaceutically acceptable salt thereof, QD on days 1-21 of a first 21-day cycle; and
(b) administering about 1200 mg of atezolizumab on day 1 of the first 21-day cycle.

54. The use of any one of claims 50 to 53, wherein a dosing regimen includes two or more cycles.

55. The use of any one of claims 47 to 54, wherein Compound 1 is administered at an amount of about 5 mg-100 mg.

56. The use of any one of claims 47 to 55, wherein Compound 1 is administered at an amount of about 5 mg, 10 mg, 20 mg, 40 mg, 60 mg, 80 mg, or 100 mg.

57. The use of any one of claims 47 to 56, wherein the lung cancer is non-small cell lung cancer (NSCLC).

58. The use of any one of claims 47 to 56, wherein the head and neck cancer is head and neck squamous cell carcinoma (HNSCC).

59. The use of any one of claims 47 to 56, wherein the melanoma is BRAFT WT melanoma.

60. A combination therapy comprising Compound 1, or a pharmaceutically acceptable salt thereof, and atezolizumab for use in the treatment of lung cancer, head and neck cancer, or melanoma.

61. The combination therapy of claim 60, wherein:
(a) Compound 1, or a pharmaceutically acceptable salt thereof, is administered QD on days 1-21 of a first 21-day cycle; and
(b) atezolizumab is administered Q3W on day 1 of the first 21-day cycle.

62. The combination therapy of claim 60 or 61, wherein:
(a) Compound 1, or a pharmaceutically acceptable salt thereof, is administered QD on days 1-21 of a first 21-day cycle; and
(b) atezolizumab is administered on day 1 of the first 21-day cycle.

63. The combination therapy of any one of claims 60 to 62, wherein:
(a) about 50 mg-500 mg of Compound 1, or a pharmaceutically acceptable salt thereof, is administered QD on days 1-21 of a first 21-day cycle; and
(b) about 1200 mg of atezolizumab is administered on day 1 of the first 21-day cycle.

64. The combination therapy of any one of claims 60 to 63, wherein a dosing regimen includes two or more cycles.

65. The combination therapy of any one of claims 60 to 64, wherein Compound 1 is administered at an amount of about 5 mg-100 mg.

66. The combination therapy of any one of claims 60 to 65, wherein Compound 1 is administered at an amount of about 5 mg, 10 mg, 20 mg, 40 mg, 60 mg, 80 mg, or 100 mg.

67. The combination therapy of any one of claims 60 to 66, wherein the lung cancer is non-small cell lung cancer (NSCLC).

68. The combination therapy of any one of claims 60 to 66, wherein the head and neck cancer is head and neck squamous cell carcinoma (HNSCC).

69. The combination therapy of any one of claims 60 to 66, wherein the melanoma is BRAFT WT melanoma.


SEQUENCE LISTING (10 sequences)

SEQ ID NO: 1 - protein, 447 aa, synthetic construct
  REGION 1..447: note = Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
  REGION 1..214: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
  REGION 1..10: note = Synthetic Construct
GFTFSDSWIH

SEQ ID NO: 4 - protein, 18 aa, synthetic construct
  REGION 1..18: note = Synthetic Construct
AWISPYGGSTYYADSVKG

SEQ ID NO: 5 - protein, 9 aa, synthetic construct
  REGION 1..9: note = Synthetic Construct
RHWPGGFDY

SEQ ID NO: 6 - protein, 11 aa, synthetic construct
  REGION 1..11: note = Synthetic Construct
RASQDVSTAVA

SEQ ID NO: 7 - protein, 7 aa, synthetic construct
  REGION 1..7: note = Synthetic Construct
SASFLYS

SEQ ID NO: 8 - protein, 9 aa, synthetic construct
  REGION 1..9: note = Synthetic Construct
QQYLYHPAT

SEQ ID NO: 9 - protein, 118 aa, synthetic construct [sequence omitted]

SEQ ID NO: 10 - protein, 108 aa, synthetic construct
  REGION 1..108: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR